(19) United States
(12) Patent Application Publication
(10) Pub. No.: US 2002/0061530 A1
(43) Pub. Date: May 23, 2002

(54) Enhanced targeting of DNA sequences by recombinase protein and single-stranded homologous DNA probes using DNA analog activation

(76) Inventors: Boris P. Belotserkovskii, Mountain View, CA (US); Gurucharan Reddy, Fremont, CA (US); David A. Zarling, Menlo Park, CA (US)

Correspondence Address: FLEHR HOHBACH TEST, ALBRITTON & HERBERT LLP, Suite 3400, Four Embarcadero Center, San Francisco, CA, 94111-4187, US

(21) Appl. No.: 09/919,345

(22) Filed: Jul. 30, 2001

Related U.S. Application Data
(60) Provisional application No. 60/222,272, filed on Jul. 31, 2000.

(51) Int. Cl. C12Q 1/68; C07H 21/04; C12P 19/34; C07K 14/00

(52) U.S. Cl. 435/6; 435/91.2; 530/350; 536/23.2

(57) ABSTRACT

the present invention is directed to methods and compositions for using DNA analog probes in increasing the efficiency of DNA targeting by recombinase coated nucleoprotein filaments. The present invention teaches novel methods and compositions that combine the traditional advantages of RecA coated filaments in catalyzing homology searches with the utility of PNA (peptide nucleic acids) to bind DNA with a very high affinity and its ability to locally open the target DNA thereby improving the kinetics of RecA-mediated strand exchange.

This application is a continuing application of U.S. Ser. No. 60/222,272.

### FIELD OF THE INVENTION

The present invention is directed to methods and compositions using DNA analog probes in activating and increasing the efficiency of DNA targeting by recombinase coated nucleoprotein filaments. This invention finds use in modifying DNA sequences in target DNA, both in vivo and in vitro. Furthermore, this invention finds use in activating homologous recombination, increasing homologous recombination frequencies and mutagenesis in target DNA. This invention also finds use in the stimulation of DNA repair enzymes to excise target DNA sequences. Additionally, this invention finds use in gene cloning, gene family cloning of target DNA sequences and in activating cloning of homologous linear genomic DNA.

### BACKGROUND OF THE INVENTION

Homologous recombination (or general recombination) is defined as the exchange of homologous recombination is that the enzymes responsible for the recombination event can presumably use any pair of homologous sequences as substrates, although some types of sequence may be favored over others. Both genetic and cytological studies have indicated that such a crossing-over process occurs between pairs of homologous chromosomes during meiosis in higher organisms.

Alternatively, in site-specific recombination, exchange occurs at a specific site, as in the integration of phage λ into the E. coli chromosome and the excision of λ DNA from it. In this case, site-specific recombination involves specific sequences of the phage DNA and bacterial DNA. Within these sequences there is only a short stretch of homology necessary for the recombination event, but not sufficient for it. The enzymes involved in this event generally cannot recombine other pairs of homologous (or nonhomologous) sequences, but act specifically on the particular phage and bacterial sequences.

Although both site-specific recombination and homologous recombination are useful mechanisms for genetic engineering of DNA sequences, targeted homologous recombination provides a basis for targeting and altering essentially any desired sequence in a duplex DNA molecule, such as targeting a DNA sequence in a chromosome for replacement by another sequence. Site-specific recombination hag been proposed as one method to integrate transfected DNA at chromosomal locations having specific recognition sites (O'Gorman et al. (1991) Science 251:1351; Onouchi et al. (1991) Nucleic Acids Res. 19:6373). Unfortunately, since this approach requires the presence of specific target sequences and recombinases, its utility for targeting recombination events at any particular chromosomal location is severely limited in comparison to targeted general recombination.

For these reasons and others, targeted homologous recombination has been proposed for treating human genetic diseases. Human genetic diseases include (1) classical human genetic diseases wherein a disease allele having a mutant genetic lesion is inherited from a parent (e.g., adenosine deaminase deficiency, sickle cell anemia, thalassemias), (2) complex genetic diseases like cancer, where the pathological state generally results from one or more specific inherited or acquired mutations, and (3) acquired genetic disease, such as an integrated provirus (e.g., hepatitis B virus). However, current methods of targeted homologous recombination are inefficient and produce desired homologous recombinants only rarely, necessitating complex cell selection schemes to identify and isolate correctly targeted recombinants.

A primary step in homologous recombination is DNA strand exchange, which involves a pairing of a DNA duplex with at least one DNA strand containing a complementary sequence to form an intermediate recombination structure containing heteroduplex DNA (see, Radding, C. M. (1982) Ann. Rev. Genet. 16:405; U.S. Pat. No. 4,888,274). The heteroduplex DNA may take several forms, including a three DNA strand containing triplex form wherein a single complementary strand invades the DNA duplex (Hsieh et al. (1990) Genes and Development 4:1951; Rao et al., (1991) PNAS 88:2984)) and, when two complementary DNA strands pair with a DNA duplex, a classical Holliday recombination joint or chi structure (Holliday, R. (1964) Genet. Res. 5:282) may form, or a double-D loop (“Diagnostic Applications of Double-D Loop Formation” U.S. Ser. No. 07/755,462, filed Sept. 4, 1991, which is incorporated herein by reference). Once formed, a heteroduplex structure may be resolved by strand breakage and exchange, so that all or a portion of an invading DNA strand is spliced into a recipient DNA duplex, adding or replacing a segment of the recipient DNA duplex. Alternatively, a heteroduplex structure may result in gene conversion, wherein a sequence of an invading strand is transferred to a recipient DNA duplex by repair of mismatched bases using the invading strand as a template (Genes, 3rd Ed. (1987) Lewin, B., John Wiley, New York, N.Y.; Lopez et al. (1987) Nucleic Acids Res. 15:5643). Whether by the mechanism of breakage and rejoining or by the mechanism(s) of gene conversion, formation of heteroduplex DNA at homologously paired joints can serve to transfer genetic sequence information from one DNA molecule to another.

The ability of homologous recombination (gene conversion and classical strand breakage/rejoining) to transfer genetic sequence information between DNA molecules makes targeted homologous recombination a powerful method in genetic engineering and gene manipulation.

The ability of mammalian and human cells to incorporate exogenous genetic material into genes residing on chromosomes has demonstrated that these cells have the general enzymatic machinery for carrying out homologous recombination required between resident and introduced sequences. These targeted recombination events can be used to correct mutations at known sites, replace genes or gene segments with defective ones, or introduce foreign genes into cells. The efficiency of such gene targeting techniques is related to several parameters: the efficiency of DNA delivery into cells, the type of DNA packaging (if any) and the size and conformation of the incoming DNA, the length and position of regions homologous to the target site (all these parameters also likely affect the ability of the incoming homologous DNA sequences to survive intracellular nuclease attack), the efficiency of recombination at particular chromosomal sites and whether recombinant events are homologous or nonhomologous. Over the past 10 years or so, several methods have been developed to introduce DNA into mammalian cells: direct needle microinjection, transfection, electroporation, electroincorporation, retroviruses, adenoviruses, adeno-associated viruses; Herpes viruses, and other viral packaging and delivery systems, polyamidoamine dendimers, liposomes, and most recently techniques using DNA-coated microprojectiles delivered with a gene gun (called a biolistics device), or narrow-beam lasers (laser-poration). The processes associated with some types of gene transfer have been shown to be both mutagenic and carcinogenic (Bardwell, (1989) Mutagenesis 4:245), and these possibilities must be considered in choosing a transfection approach.

The choice of a particular DNA transfection procedure depends upon its availability to the researcher, the technique's efficiency with the particular chosen target cell type, and the researchers concerns about the potential for generating unwanted genome mutations. For example, retroviral integration requires dividing cells, always results in nonhomologous recombination events, and retroviral insertion within a coding sequence of nonhomologous (i.e., non-targeted) gene could cause cell mutation, by inactivating the gene's coding sequence (Friedmann, (1989) Science 244:1275). Newer retroviral-based DNA delivery systems are being developed using defective retroviruses. However, these disabled viruses must be packaged using helper systems, are often obtained at low titer, and recombination is still not site-specific, thus recombination between endogenous cellular retrovirus sequences and disabled virus sequences could still produce wild-type retrovirus capable of causing gene mutation. Adeno- or polyoma virus based delivery systems appear very promising (Samulski et al., (1991) EMBO J. 10:2941; Gareis et al., (1991) Cell. Molec. Biol. 37:191; Rosenfeld et al. (1992) Cell 68:143) although they still require specific cell membrane recognition and binding characteristics for target cell entry. Liposomes often show a narrow spectrum of cell specificities, and when DNA is coated externally on to them, the DNA is often sensitive to cellular nucleases. Newer polycationic lipospermines compounds exhibit broad cell ranges (Behr et al., (1989) Proc. Natl. Acad. Sci. USA 86:6982) and DNA is coated by these compounds. In addition, a combination of neutral and cationic lipid has been shown to be highly efficient at transfection of animal cells and showed a broad spectrum of effectiveness in a variety of cell lines (Rose et al., (1991) BioTechniques 10:520). Galactosylated bis-acridine has also been described as a carrier for delivery of polynucleotides to liver cells (Haensler J L and Szoka F C (1992), Abstract V211 in J. Cell. Biochem. Supplement 16F, Apr. 3-16, 1992, incorporated herein by reference). Electroporation also appears to be applicable to most cell types. The efficiency of this procedure for a specific gene is variable and can range from about one event per 3×10⁴ transfected cells (Thomas and Capecchi, (1987) Cell 51:503) to between one in 10⁷ and 10⁸ cells receiving the exogenous DNA (Koller and Smithies, (1989) Proc. Natl. Acad. Sci. (U.S.A.) 86:8932). Microinjection of exogenous DNA into the nucleus has been reported to result in a high frequency of stable transfected cells. Zimmer and Gruss (Zimmer and Gruss (1989) Nature 338:150) have reported that for the mouse hox1.1 gene, 1 per 150 microinjected cells showed a stable homologous site specific alteration.

Several methods have been developed to detect and/or select for targeted site-specific recombinants between vector DNA and the target homologous chromosomal sequence (see, Capecchi, (1989) Science 244:1288 for review). Cells which exhibit a specific phenotype after site-specific recombination, such as occurs with alteration of the HPRT gene, can be obtained by direct selection on the appropriate growth medium. Alternatively, a selective marker sequence such as neo can be incorporated into a vector under promoter control, and successful transfection can be scored by selecting G418^{r} cells followed by PCR to determine whether neo is at the targeted site (Joyner et al., (1989) Nature 338:153). A positive-negative selection (PNS) procedure using both neo and HSV-tk genes allows selection for transfectants and against nonhomologous recombination events, and significantly enriched for desired disruption events at several different mouse genes (Mansour et al., (1988) Nature 336:348). This procedure has the advantage that the method does not require that the targeted gene be transcribed. If the targeted gene is transcribed, a promoter-less marker gene can be incorporated into the targeting construct so that the gene becomes activated after homologous recombination with the target site (Jasin and Berg, (1988) Genes and Development 2:1353; Doetschman et al. (1988) Proc. Natl. Acad. Sci. (U.S.A.) 85:8583; Dorini et al., (1989) Science 243:1357; Itzhaki and Porter, (1991) Nucl. Acids Res. 19:3835). Recombinant products produced using vectors with selectable markers often continue to retain these markers as foreign genetic material at the site of transfection, although loss does occur. Valancius and Smithies (Valancius and Smithies, (1991) Molec. Cellular Biol. 11:1402) have described an “in-out” targeting procedure that allowed a subtle 4-bp insertion modification of a mouse HPRT target gene. The resulting transfectant contained only the desired modified gene sequence and no selectable marker remained after the “out” recombination step. Co-transformation of cells with two different vectors, one vector contained a selectable gene and the other used for gene disruption, increases the efficiency of isolating a specific targeting reaction (Reid et al., (1991) Molec. Cellular Biol. 11:2769) among selected cells that are subsequently scored for stable recombinants.

Unfortunately, exogenous sequences transferred into eukaryotic cells undergo homologous recombination with homologous endogenous sequences only at very low frequencies, and are so inefficiently recombined that large numbers of cells must be transfected, selected, and screened in order to generate a desired correctly targeted homologous recombinant (Kucherlapati et al. (1984) Proc. Natl. Acad. Sci. (U.S.A.) 81:3153; Smithies, 0. (1985) Nature 317:230; Song et al. (1987) Proc. Natl. Acad. Sci. (U.S.A.) 84:6820; Doetschman et al. (1987) Nature 330:576; Kim and Smithies (1988) Nucleic Acids Res. 16:8887; Doetschman et al. (1988) op.cit. Koller and Smithies (1989) op.cit.; Shesely et al. (1991) Proc. Natl. Acad. Sci. (U.S.A.) 88:4294; Kim et al. (1991) Gene 103:227, which are incorporated herein by reference).

Koller et al. (1991) Proc. Natl. Acad. Sci. (U.S.A.), 88:10730 and Snouwaert et al. (1992) Science 257:1083, have described targeting of the mouse cystic fibrosis transmembrane regulator (CFTR) gene for the purpose of inactivating, rather than correcting, a murine CFTR allele. Koller et al. employed a large (7.8kb) homology region in the double-stranded DNA targeting construct, but nonetheless reported a low frequency for correct targeting (only 1 of 2500 G418-resistant cells were correctly targeted). Thus, even targeting constructs having lone homology regions are inefficiently targeted.

Several proteins or purified extracts having the property of promoting homologous recombination (i.e., recombinase activity) have been identified in prokaryotes and eukaryotes (Cox and Lehman (1987) Ann. Rev. Biochem. 56:229; Radding, C. M. (1982) op.cit.; Madiraju et al. (1988) Proc. Natl. Acad. Sci. (U.S.A.) 85:6592; McCarthy et al. (1988) Proc. Natl. Acad. Sci. (U.S.A.) 85:5854; Lopez et al. (1987) op.cit., which are incorporated herein by reference). These general recombinases presumably promote one or more steps in the formation of homologously-paired intermediates, strand-exchange, gene conversion, and/or other steps in the process of homologous recombination.

The frequency of homologous recombination in prokaryotes is significantly enhanced by the presence of recombinase activities. Several purified proteins catalyze homologous pairing and/or strand exchange in vitro, including: E. coli RecA protein, the T4 uvsX protein, and the rec1 protein from Ustilago maydis. Recombinases, like the RecA protein of E. coli are proteins which promote strand pairing and exchange. The most studied recombinase to date has been the RecA recombinase of E. coli, which is involved in homology search and strand exchange reactions (see, Cox and Lehman (1987) op.cit.). RecA is required for induction of the SOS repair response, DNA repair, and efficient genetic recombination in E. coli. RecA can catalyze homologous pairing of a linear duplex DNA and a homologous single strand DNA in vitro. In contrast to site-specific recombinases, proteins like recA which are involved in general recombination recognize and promote pairing of DNA structures on the basis of shared homology, as has been shown by several in vitro experiments (Hsieh and Camerini-Otero (1989) J. Biol. Chem. 264:5089; Howard-Flanders et al. (1984) Nature 309:215; Stasiak et al. (1984) Cold Spring Harbor Symp. Quant. Biol. 49:561; Register et al. (1987) J. Biol. Chem. 262:12812). Several investigators have used recA protein in vitro to promote homologously paired triplex DNA (Cheng et al. (1988) J. Biol. Chem. 263:15110; Ferrin and Camerini-Otero (1991) Science 354:1494; Ramdas et al. (1989) J. Biol Chem. 264:11395; Strobel et al. (1991) Science 354:1639; Hsieh et al. (1990) op.cit.; Rigas et al. (1986) Proc. Natl. Acad. Sci. (U.S.A.) 83:9591; and Camerini-Otero et al. U.S. Pat. No. 7,611,268 (available from Derwent), which are incorporated herein by reference. Unfortunately many important genetic engineering manipulations involving homologous recombination, such as using homologous recombination to alter endogenous DNA sequences in a living cell, cannot be done in vitro. Further, gene therapy requires highly efficient homologous recombination of targeting vectors with predetermined endogenous target sequences, since selectable marker selection schemes, such as those currently available in the art, are not usually practicable in human beings. In addition, the yields of these multistranded DNA-DNA hybrids using recombinase enzymes may vary in different systems, and, in general, may produce less than 100% yields, especially after deproteinization.

PNA (peptide nucleic acid) is a modified DNA analog, which contains a peptide-like backbone instead of a phosphodiester backbone (Nielsen et al., Science, 254, 497-1500 (1991)). The advantage of PNA probes is that under appropriate conditions they can be targets to specific sequences within linear or superhelical DNA with near 100% efficiency. Because of its superior DNA and RNA binding properties, PNA has numerous application for gene regulation at the level of transcription and translation (Nielson et al., Curr. Opin. Biotechnol., 10, 71-75 (1996)) for rare cutting of genomic DNA (Veselkov et al, Nuc. Acid Res., 24(14), 2483-7, (1996)), for DNA labeling, mapping and isolation (Demidov et al., 1994, Nuc. Acids Res., 22, 5218-5222 (1994), and for DNA mutagenesis (Faruqi et al., Porc. Natl. Acad. Sci. USA, 95, 1398-1403 (1998)).

It is attractive and part of the present invention herein to combine the very high DNA hybridizing activity and stability of PNA and the recombinogenecity of RecA protein coated nucleic acid probes in the same targeting reaction.

Thus, there exists a need in the art for methods of efficiently altering predetermined endogenous genetic sequences by homologous pairing and homologous recombination in vivo by introducing one or more exogenous targeting polynucleotide(s) that efficiently and specifically homologously pair with a predetermined endogenous DNA sequence. There also exists a need in the art to increase homologous recombination frequencies. There exists a need in the art for high-efficiency gene targeting, so as to avoid complex in vitro selection protocols (e.g., neo gene selection with G418), which are of limited utility for In vivo gene therapy on affected individuals.

### SUMMARY OF THE INVENTION

In accordance with the objects outlined above, the present invention provides compositions comprising an analog probe and a first recombinase coated single stranded nucleic acid probe for targeting DNA. In one aspect, the single stranded nucleic acid probe is DNA.

In another aspect, the present invention provides a composition comprising, in addition to the above composition, a second recombinase coated single stranded nucleic acid probe which is substantially complementary to the first probe.

In one aspect, the recombinase used is a prokaryotic recombinase, including but not limited to, RecA recombinase. In another aspect, the recombinase used is a eukaryotic recombinase, including but not limited to, Rad51 recombinase. In an additional aspect, a complex of recombinase proteins are used.

In one aspect, the compositions comprise analog probes which include, but are not limited to, peptide nucleic acid (PNA), N3′—P5′ phosphoramidate nucleic acids (NP), 2′-O-methoxyethyl nucleic acids, 2′-fluoro-arabino nucleic acids or other analogs described below. In a preferred embodiment, the analog probe used comprises PNA.

In one aspect, the invention provides compositions wherein the analog probe is a fusion sequence comprising nucleoside analogs and naturally occurring nucleosides. In another aspect, the nucleoside analog comprises at least one peptide nucleoside.

In one aspect, the present invention provides methods for enhancing targeting of DNA sequences, said method comprising providing a sample containing a double stranded nucleic acid target sequence, an analog probe for activating this nucleic acid, and a first recombinase coated single stranded nucleic acid probe comprising a homology clamp that is substantially complementary to one strand of the target nucleic acid sequence and wherein, said recombinase coated single stranded probe hybridizes to said target sequence.

In an additional aspect, the above method additionally comprises a second recombinase coated single stranded nucleic acid probe that is also substantially complementary to the first single stranded nucleic acid probe and additionally, hybridizes to said double stranded target nucleic acid.

In one aspect, the invention provides a method wherein at least one of said first and second probes comprise at least one alteration as compared to said target sequence and wherein the method further alters the target sequence by homologous recombination with at least one of said probes. In another aspect, both the first and second recombinase coated single stranded nucleic acid have the alteration. The alteration can be a substitution mutation, a deletion mutation or an insertion mutation.

In one aspect, the invention provides a method for the activating analog probe to create a nucleation site for hybridization of a first recombinase coated single stranded nucleic acid to the target sequence.

In one aspect, the nucleation site can be a single D-loop or a double D-loop structure.

In one aspect, the invention provides a method for the activating analog probe to form a hybridization complex with a portion of said target nucleic acid. In another aspect, the hybridization complex formed is at least as stable as, a non-analog nucleic acid hybridization complex.

In one aspect, the invention provides a method for the activating analog probe to facilitate production of double stranded gaps in the target nucleic acid sequence which are then repaired by recombinase coated single stranded probes.

In one aspect, the invention provides methods wherein the first and second recombinase coated probes comprise a purification tag which enables separation of the probe and target utilizing the tag. In a further aspect, the purification tag comprises biotin.

In another aspect, the method encompasses inserting the target sequence into a cloning vector.

### BRIEF DESCRIPTION OF THE DRAWINGS

FIG. 1A. Analog probe-induced targeting by RecA-coated probes: Activation occurs by the creation of a nucleation site by the analog probe within the target DNA. This local opening within the target DNA improves the kinetics of RecA-mediated strand exchange. Here, the RecA-coated complementary single-stranded probes contain a heterologous insertion sequence depicted as thick lined loops.

FIG. 1B. Stabilization of single D-loop hybrids by analog probes: When an analog probe binds within the probe-target hybrid, the analog probe stabilizes the single D-loops formed by trapping the strand-exchange process or dissociation of the hybrids.

FIG. 1C. Stabilization of double D-loop hybrids by analog probes: Analog probes like PNA cause activation of the target by the creation of a nucleation site within the “closed” D-loop and this structure improves the kinetics for a second incoming RecA-coated single stranded probe. Analog probes stabilize the double D-loops formed in their “opened” state and prevent dissociation of the hybrids.

FIG. 2. Analog probe-directed double-stranded break in target DNA: After activation, which involves target DNA opening by an analog probe like PNA, the single stranded regions in the PNA-DNA complex can be digested by single-strand DNA-specific endonucleases or junction specific endonucleases resulting in a double-stranded gap in the target DNA. Then, through RecA mediated strand exchange, single strand probes comprising heterologous inserts or deletions are used to introduce changes in the target DNA. Thus, analog probe treated cells have increased homologous recombination frequencies within and around sites occupied by the analog-DNA complex. The thick lines in the single stranded probes are non-homologous with respect to target DNA, therefore are insertion sequences.

FIG. 3A. Analog probe-directed DNA excision and repair: Since analog probes create local DNA distortions, either by structural distortions or by transcriptional arrest, this can result in stimulation of DNA repair enzymes. Shown in this figure are possible pathways for processing multistranded PNA-DNA hybrid. The thick lined loops or lines depict non-homologous (with respect to the target DNA) insertion sequences.

FIG. 3B. Analog probe-directed DNA excision and repair: During DNA replication, analog probes create local DNA distortions thereby constraining DNA copying, and induces DNA repair at such sites. Shown in this figure are possible pathways for processing multistranded PNA-DNA hybrids formed during DNA replication. The thick lined loops or lines depict non-homologous (with respect to the target DNA) insertion sequences.

FIG. 3C. Analog probe-directed DNA excision and repair when PNA site is inside heterologous insert site: Example of analog probes disrupting DNA replication wherein the analog probe binding site is inside the heterologous insert site. The thick lined loops or lines depict non-homologous (with respect to the target DNA) or heterologous insertion sequences.

FIG. 4. Cloning of DNA fragments mediated by PNA analog probes.

FIG. 5A. Scheme for targeting the human HPRT gene fragment with PNA analog probes. Shown here are the probes 1-2 and 1-3 obtained by PCR. Probe 1-2 has three bp overlap withe the PNA binding site. Probe 1-3 contains the PNA binding site within it.

FIG. 5B. Targeting of human HPRT gene fragment with PNA analog probes. Lane 1 shows that the presence of PNA analog probe 1-2 strongly increases the yield of the hybrids. (See Example for details).

FIG. 5C. Targeting of human HPRT gene fragment with PNA analog probes. Lane 1 shows that the presence of PNA analog probe 1-3 strongly increases the yield of the hybrids. (See Example for details).

### DETAILED DESCRIPTION OF THE INVENTION

This invention is directed to the use of analog probes to assist recombinase coated single stranded nucleic acid probe hybridization to nucleic acid target sequences. The invention is based on the fact that many nucleic acid analogs, such as peptide nucleic acids (PNAs), form hybridization complexes with higher melting temperatures (Tm's) than naturally occurring nucleic acid complexes. Essentially, the analog probe will preferentially invade a double stranded nucleic acid duplex target sequence, thus forming a nucleation site (e.g. a “bubble” or “opening”) that then allows the recombinase coated single stranded nucleic acid probes of the invention to bind to the nucleic acid target sequence. Thus, analog probe hybridization facilitates subsequent hybridization of recombinase coated single stranded nucleic acid probes. The recombinase enzyme catalyzes the pairing and strand exchange between the target nucleic acid sequence and the single stranded nucleic acid probe. This pairing leads to the formation of multistranded nucleic acid hybrids. The multistrand hybrids may serve as intermediates in recombination events resulting in sequence deletion, insertion or the creation of mutant sequences. In addition, by using purification tags on either the recombinase coated single stranded nucleic acid probes or the analog probes, the multistrand hybrids may serve as a means for detecting or isolating and cloning the nucleic acid target sequence. Accordingly, the present invention provides methods and compositions for the detection, isolation and alteration of target sequences in a sample. The invention draws on technology outlined in U.S. Pat. Nos. 5,763,240; 6,200,812; 6,074,853; PCT/US Ser. No. 93/03868; PCT/US Ser. No. 98/05223; WO Ser. No. 99/60108; WO Ser. No. 99/37755; WO Ser. No. 00/09755; WO Ser. No. 00/56872; WO Ser. No. 00/63365; PCT/US Ser. No. 00/35666; PCT/US Ser. No. 00/04592 all of which are expressly incorporated by reference.

As will be appreciated by those in the art, the sample or sample solution may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is known in the art, such as PCR amplification reaction); purified samples, such as purified genomic DNA, RNA, etc.; raw samples (bacteria, virus, genomic DNA, etc.); as will be appreciated by those in the art, virtually any experimental manipulation may have been done on the sample.

The present invention provides compositions and methods for detecting the presence or absence of nucleic acid target sequences in a sample. By “nucleic acid” or “oligonucleotide” or grammatical equivalents herein means at least two nucleotides covalently linked together. A nucleic acid of the present invention will generally contain phosphodiester bonds, although in some cases, as outlined below, for example for the creation of nucleic acid analog probes, nucleic acid analogs, as further outlined below, are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nuc. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); those with bicyclic structures including locked nucleic acids, Koshkin et al., J. Am. Chem. Soc. 120:13252-3 (1998); non-ionic backbones (U.S. Pat. Nos. 5,386,023, 5,637,684, 5,602,240, 5,216,141 and 4,469,863; Kiedrowshi et al., Angew. Chem. Intl. Ed. English. 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Nucleic acids containing 2′ substitutions, such as 2′-O-methoxyethyl (Baker, B. F. et al. J. Biol. Chem. 272, 11994-12000 (1997)) and 2′-fluoro-arabinonucleic acid (Damha, M. J. et al. J. Am. Chem. Soc. 120, 13545 (1998)) are also included in the definition of nucleic acid. Several nucleic acid analogs are described in Rawls, C & E News Jun. 2, 1997 page 35. All of these references are hereby expressly incorporated by reference. These modifications of the ribose-phosphate backbone may be done to increase the stability and half-life of such molecules in physiological environments.

The term “nucleic acid target sequence” or “target nucleic acid” or grammatical equivalents herein means a nucleic acid sequence on a single strand of nucleic acid. The target sequence may be a portion of a gene, a regulatory sequence, genomic DNA, cDNA, RNA including mRNA and rRNA, or others. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to detect, isolate, or alter the target sequence in a sample. Generally speaking, this term will be understood by those skilled in the art. The nucleic acid target sequence may also be comprised of different target domains; for example, a first target domain of the sample target sequence may hybridize to a capture probe or a portion of capture extender probe, a second target domain may hybridize to a portion of an amplifier probe, a label probe, or a different capture or capture extender probe, etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

Nucleic acid target sequences can include but are not limited to chromosomal sequences (e.g., structural genes, regulatory sequences including promoters and enhancers, recombinatorial hotspots, repeat sequences, integrated proviral-sequences, hairpins, palindromes), episomal or extrachromosomal sequences (e.g., replicable plasmids or viral replication intermediates) including chloroplast and mitochondrial DNA sequences.

The nucleic acid target sequence may be single stranded or double stranded, as specified, or contain portions of both double stranded or single stranded sequence. The nucleic acid target sequence may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996), both of which are hereby incorporated by reference.

The term “naturally-occurring” as used herein as applied to an object refers to the fact that an object can be found in nature. For example, a polynucleotide sequence that is present in an organism (including viruses) that can be isolated from a source in nature and which has not been intentionally modified by man in the laboratory is naturally-occurring.

In some embodiments, the nucleic acid target sequence is a disease allele. As used herein, the term “disease allele” refers to an allele of a gene which is capable of producing a recognizable disease. A disease allele may be dominant or recessive and may produce disease directly or when present in combination with a specific genetic background or pre-existing pathological condition. A disease allele may be present in the gene pool or may be generated de novo in an individual by somatic mutation. For example and not limitation, disease to alleles include: activated oncogenes, a sickle cell anemia allele, a Tay-Sachs allele, a cystic fibrosis allele, a Lesch-Nyhan allele, a retinoblastoma-susceptibility allele, a Fabry's disease allele, and a Huntington's chorea allele. As used herein, a disease allele encompasses both alleles associated with human diseases and alleles associated with recognized veterinary diseases. For example, the ΔF508 CFTR allele in a human disease allele which is associated with cystic fibrosis in North Americans.

The invention further provides hybridizing analog probes to some portion of the nucleic acid target sequence. By “analog probe” herein is meant nucleic acids containing modifications to the natural-occurring phosphodiester linkages or modifications to the natural occurring ribose backbone. In addition, analog probes hybridize to complementary nucleic acid sequences at least as well as the corresponding natural occurring nucleic acids. Examples of analog probes include but are not limited to peptide nucleic acids (PNA) (Nielson et al., Curr. Opin. Biotechnol., 10, 71-75 (1996); Demidov et al., 1994, Nuc. Acids Res., 22, 5218-5222 (1994)), N3′—P5′ phosphoramidate nucleic acids (NP), (Faria, M. et al., Nat. Biotechnol. 19, 40-44 (2001)), 2′-O-methoxyethyl nucleic acids (Baker, B. F. et al., J. Biol. Chem., 272, 11994-12000 (1997)), and 2′-fluoro-arabino nucleic acids (Damha, M. J. et al., J. Am. Chem. Soc., 120, 13545 (1998)), and others cited above.

Preferred analog probes include those that form duplexes with naturally occurring nucleic acids (particularly DNA and RNA) with melting temperatures (Tm's) higher than that of the naturally occurring duplexes. Particularly preferred analog probes comprise at least a portion comprising PNA monomers.

In a preferred embodiment, the analog probe does not contain any linkages that are the natural-occurring linkages.

In a preferred embodiment, the analog probe comprises both the natural-occurring phosphodiester linkages and linkages that are not the natural-occurring linkages. Thus, for example, analog probes with a first domain of DNA and a second domain of PNA can be made. Similarly, three domain probes can be made with mixtures of analogs and naturally occurring linkages. For example, analog probes with a first domain of DNA, a second domain of PNA and a third domain of NP can be made. In this way, as would be appreciated in the art, further combinations of natural-occurring phosphodiester linkages and linkages that are not the natural-occurring linkages can be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tm for an internal mismatch. With the non-ionic PNA backbone, the drop is closer to 7-9° C. This allows for better detection of mismatches. Though PNA has superior binding properties, it can not efficiently mediate the insertion of new genetic information into a target gene because it is not a substrate for cellular nucleic acid copying enzymes.

Analog probes are generally at least about 6 to 30 nucleotides long, preferably about 7 to 20 nucleotides long, at least about 7 to 15 nucleotides long, more preferably at least about 6 to 10 nucleotides long, or longer. The length of homology may be selected at the discretion of the practitioner on the basis of the sequence composition and complexity of the predetermined endogenous target DNA sequence(s) and guidance provided in the art, which generally indicates that 1.3 to 6.8 kilobase segments of homology are preferred when non-recombinase mediated methods are utilized (Hasty et al. (1991) Molec. Cell. Biol: 11: 5586; Shulman et al. (1990) Molec. Cell. Biol. 10:4466, which are incorporated herein by reference).

In a preferred embodiment, the sequence of the analog probe is sufficiently complimentary to the target sequence. The term “sufficiently complimentary” as used herein denotes a sequence that allows the recombinase coated probes to bind more easily to the target sequence, as more fully described below. Thus, the analog probe sequence must merely be sufficiently complimentary so as to assist recombinase coated single stranded nucleic acid probe hybridization to the target sequences.

In a preferred embodiment, the analog probe sequence is sufficiently complementary to some portion of one strand of a double stranded nucleic acid target sequence.

In a preferred embodiment, the analog probe sequence is sufficiently complementary to some portion of both strands of a double stranded nucleic acid target sequence. For example, a single analog probe simultaneously binds to both strands of the double stranded target sequence, allowing the recombinase coated single stranded nucleic acid probe to more easily hybridize with the target sequence.

In a preferred embodiment, the analog probe sequence sufficiently corresponds to a sequence within the vicinity of the nucleic acid target sequence. The term “within the vicinity” as used herein means generally from about 0 to 10 nucleotides from the 5′ or 3′ end of the nucleic acid target sequence. As explained above, the analog probe sequence may be designed to bind a single strand of a sequence in the vicinity of a nucleic acid target sequence or to both strands of a sequence in the vicinity of a nucleic acid target sequence simultaneously. The target sequence can also be inside the target sequence.

In another preferred embodiment, the analog probe sequence sufficiently corresponds to a sequence within the nucleic acid target sequence.

As outlined further below, analog probes can comprise purification tags, cell targeting components, separation moieties, epitope tags, separation sequences and/or chemical substituents.

By “single stranded nucleic acid probes” herein is meant the polynucleotides used to clone, alter, or detect the target nucleic acids as described herein. Single stranded nucleic acid probes are generally one or most preferably two substantially complementary single-stranded DNAs.

Single stranded nucleic acid probes are generally at least about 2 to 100 nucleotides long, preferably at least about 5- to 100 nucleotides long, at least about 250 to 500 nucleotides long, more preferably at least about 500 to 2000 nucleotides long, or longer; however, as the length of a single stranded nucleic acid probe increases beyond about 20,000 to 50,000 to 400,000 nucleotides, the efficiency or transferring an intact single stranded nucleic acid probe into the cell decreases. The length of homology may be selected at the discretion of the practitioner on the basis of the sequence composition and complexity of the predetermined endogenous target DNA sequence(s) and guidance provided in the art, which generally indicates that 1.3 to 6.8 kilobase segments of homology are preferred (Hasty et al. (1991) Molec. Cell. Biol. 11:5586; Shulman et al. (1990) Molec. Cell. Biol. 10:4466, which are incorporated herein by reference). Single stranded nucleic acid probes have at least one sequence that substantially corresponds to, or is substantially complementary to, a predetermined endogenous DNA sequence (i.e., a DNA sequence of a polynucleotide located in a target cell, such as a chromosomal, mitochondrial, chloroplast, viral, episomal, or mycoplasmal polynucleotide). Such single stranded nucleic acid probe sequences serve as templates for homologous pairing with the predetermined endogenous sequence(s), and are also referred to herein as homology clamps. In single stranded nucleic acid probes, such homology clamps are typically located at or near the 5′ or 3′ end, preferably homology clamps are internally or located at each end of the polynucleotide (Bernstein et al. (1992) Molec. Cell. Biol. 12:360, which is incorporated herein by reference). Without wishing to be bound by any particular theory, it is believed that the addition of recombinases permits efficient gene targeting with single stranded nucleic acid probes having short (i.e., about 50 to 1000 base pairs long) segments of homology, as well as with single stranded nucleic acid probes having longer segments of homology.

Single stranded nucleic acid probes have at least one sequence that substantially corresponds to, or is substantially complementary to, the nucleic acid target sequence. The terms “substantially corresponds to” or “substantial identity” or “homologous” as used herein denotes a characteristic of a nucleic acid sequence, wherein a nucleic acid sequence has at least about 50 percent sequence identity as compared to a reference sequence, typically at least about 70 percent sequence identity, and preferably at least about 85 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 18 nucleotides long, typically at least about 30 nucleotides long, and preferably at least about 50 to 100 nucleotides long. “Substantially complementary” as used herein refers to a sequence that is complementary to a sequence that substantially corresponds to a reference sequence. In general, targeting efficiency increases with the length of the single stranded nucleic acid probe portion that is substantially complementary to a reference sequence present in the target DNA.

These corresponding/complementary sequences are sometimes referred to herein as “homology clamps”, as they serve as templates for homologous pairing with the nucleic acid target sequence(s). Thus, a “homology clamp” is a portion of the single stranded nucleic acid probe that can specifically hybridize to a portion of a nucleic acid target sequence. “Specific hybridization” is defined herein as the formation of hybrids between a single stranded nucleic acid probe (e.g., a polynucleotide of the invention which may include substitutions, deletion, and/or additions as compared to the predetermined target nucleic acid sequence) and a nucleic acid target nucleic acid, wherein the single stranded nucleic acid probe preferentially hybridizes to the nucleic acid target nucleic acid such that, for example, at least one discrete band can be identified on a Southern blot of nucleic acid prepared from target cells that contain the nucleic acid target sequence, and/or a single stranded nucleic acid probe in an intact nucleus localizes to a discrete chromosomal location characteristic of a unique or repetitive sequence. It is evident that optimal hybridization conditions will vary depending upon the sequence composition and length(s) of the single stranded nucleic acid probe(s) and target(s), and the experimental method selected by the practitioner. Various guidelines may be used to select appropriate hybridization conditions (see, Maniatis et al., Molecular Cloning: A Laboratory Manual (1989), 2nd Ed., Cold Spring Harbor, N.Y. and Berger and Kimmel, Methods in Enzymology, Volume 152, Guide to Molecular Cloning Techniques (1987), Academic Press, Inc., San Diego, Calif.), which are incorporated herein by reference. Methods for hybridizing a single stranded nucleic acid probe to a discrete chromosomal location in intact nuclei are known in the art, see for example WO Ser. No. 93/05177 and Kowalczykowski and Zarling (1994) in Gene Targeting, Ed. Manuel Vega.

In single stranded nucleic acid probes, such homology clamps are typically located at or near the 5′ or 3′ end, preferably homology clamps are internal or located at each end of the polynucleotide (Berinstein et al. (1992) Molec, Cell. Biol. 12:360, which is incorporated herein by reference). Without wishing to be bound by any particular theory, it is believed that the addition of recombinases permits efficient gene targeting with single stranded nucleic acid probes having short (i.e., about 10 to 1000 base pairs long) segments of homology, as well as with single stranded nucleic acid probes having longer segments of homology.

Therefore, it is preferred that single stranded nucleic acid probes of the invention have homology clamps that are highly homologous to the nucleic acid target sequence(s). Typically, single stranded nucleic acid probes of the invention have at least one homology clamp that is at least about 18 to 35 nucleotides long, and it is preferable that homology clamps are at least about 20 to 100 nucleotides long, and more preferably at least about 400-500 nucleotides long, although the degree of sequence homology between the homology clamp and the nucleic acid target sequence and the base composition of the nucleic acid target sequence will determine the optimal and minimal clamp lengths (e.g., G-C rich sequences are typically more thermodynamically stable and will generally require shorter clamp length). Therefore, both homology clamp length and the degree of sequence homology can only be determined with reference to a particular predetermined sequence, but homology clamps generally must be at least about 10 nucleotides long and must also substantially correspond or be substantially complementary to a predetermined target sequence. Preferably, a homology clamp is at least about 10, and preferably at least about 50 nucleotides long and is substantially identical to or complementary to a predetermined target sequence.

Single stranded nucleic acid probes may be produced by any number of different methods, as will be appreciated by those in the art, including, but not limited to, chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Single stranded nucleic acid probes are generally ssDNA or dsDNA, most preferably two complementary single-stranded DNAs as is more fully outlined below.

In a preferred embodiment, single stranded nucleic acid probes may be made, as will be appreciated by those in the art, after a target nucleic acid gene family or consensus sequence is selected. For example, for large single stranded nucleic acid probes, plasmids are engineered to contain an appropriately sized gene sequence with a deletion or insertion in the gene of interest and at least one flanking homology clamp which substantially corresponds or is substantially complementary to an endogenous target DNA sequence. Vectors containing a single stranded nucleic acid probe sequence are typically grown in E. coli and then isolated using standard molecular biology methods.

Alternatively, single stranded nucleic acid probes may be prepared by oligonucleotide synthesis methods, which may first require, especially with larger single stranded nucleic acid probes, formation of subfragments of the single stranded nucleic acid probe, typically followed by splicing of the subfragments together, typically by enzymatic ligation. In general, as will be appreciated by those in the art, single stranded nucleic acid probes may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. When using microinjection procedures it may be preferable to use a transfection technique with linearized sequences containing only modified target gene sequence and without vector or selectable sequences. The modified gene site is such that a homologous recombinant between the exogenous single stranded nucleic acid probe and the endogenous DNA target sequence can be identified by using carefully chosen primers and PCR, followed by analysis to detect if PCR products specific to the desired targeted event are present (Erlich et al., (1991) Science 252:1643, which is incorporated herein by reference). Several studies have already used PCR to successfully identify and then clone the desired transfected cell lines (Zimmer and Gruss, (1989) Nature 338:150; Mouellic et al., (1990) Proc. Natl. Acad. Sci. USA 87:4712; Shesely et al., (1991) Proc. Natl. Acad. Sci. USA 88:4294, which are incorporated herein by reference). This approach is very effective when the number of cells receiving exogenous single stranded nucleic acid probe (s) is high (i.e., with microinjection, or with liposomes) and the treated cell populations are allowed to expand to cell groups of approximately 1×10⁴ cells (Capecchi, (1989) Science 244:1288). When the target gene is not on a sex chromosome, or the cells are derived from a female, both alleles of a gene can be targeted by sequential inactivation (Mortensen et al., (1991) Proc. Natl. Acad. Sci. USA 88:7036). Alternatively, animals heterologous for the target gene can be bred to homologously as is known in the art.

In a preferred embodiment, a single stranded nucleic acid probe is a DNA strand, or derived by denaturation of a duplex DNA, which is substantially complementary to one (or both) strand(s) of the nucleic acid target sequence. Thus, one of the substantially complementary single stranded nucleic acid probes is substantially complementary to one strand of the nucleic acid target sequence (i.e. Watson) and the other substantially complementary single stranded single stranded nucleic acid probe is substantially complementary to the other strand of the nucleic acid target sequence (i.e. Crick). The consensus homology clamp sequence preferably contains at least 90-95% sequence homology with the target sequence (although as outlined above, less sequence homology can be tolerated), to insure sequence-specific targeting of the single stranded nucleic acid probe to the target sequence.

In a preferred embodiment, degenerate single stranded nucleic acid probes are made to encode a protein consensus sequence, as is well known in the art and described in WO Ser. No. 99/37755, incorporated by reference herein. The protein sequence is encoded by DNA triplets which are deduced using standard tables. In some cases additional degeneracy is used to enable production in one oligonucleotide synthesis. In many cases motifs are chosen to minimize degeneracy. In addition, the consensus sequences may be designed to facilitate amplification of neighboring sequences. This can utilize two motifs as indicated by faithful or error prone amplification. Alternatively outside sequences can be used as is indicated using vector sequence. In addition degenerate oligonucleotides can be synthesized and used directly in the procedure without amplification.

In a preferred embodiment, the single stranded nucleic acid probe and/or the analog probe is a consensus nucleic acid sequence. By “consensus sequence” herein is meant an amino acid consensus sequence of a gene family. By “consensus nucleic acid sequence” herein is meant a nucleic acid that encodes a consensus protein sequence of a functional domain of a gene family. In addition, “consensus nucleic acid sequence” can also refer to cis sequences that are non-coding but can serve a regulatory or other role. As outlined below, generally a library of consensus nucleic acid sequences are used, that comprises a set of degenerate nucleic acids encoding the protein consensus sequence. A wide variety of protein consensus sequences for a number of gene families are known. A “gene family” therefore is a set of genes that encode proteins that contain a functional domain for which a consensus sequence can be identified. However, in some instances, a gene family includes non-coding sequences; for example, consensus regulatory regions can be identified. For example, gene family/consensus sequences pairs are known for the G-protein coupled receptor family, the AAA-protein family, the bZIP transcription factor family, the mutS family, the recA family, the Rad51 family, the dmel family, the recF family, the SH2 domain family, the Bcl-2 family, the single-stranded binding protein family, the TFIID transcription family, the TGF-beta family, the TNF family, the XPA family, the XPG family, actin binding proteins, bromodomain GDP exchange factors, MCM family, ser/thr phosphatase family, etc.

As will be appreciated by those in the art, the proteins of the gene families generally do not contain the exact consensus sequences; generally consensus sequences are artificial sequences that represent the best comparison of a variety of sequences. The actual sequence that corresponds to the functional sequence within a particular protein is termed a “consensus functional domain” herein; that is, a consensus functional domain is the actual sequence within a protein that corresponds to the consensus sequence. A consensus functional domain may also be a “predetermined endogenous DNA sequence” (also referred to herein as a “predetermined nucleic acid target sequence”) that is a polynucleotide sequence contained in a target cell. An exogenous polynucleotide is a polynucleotide which is transferred into a target cell. By “predetermined” or “pre-selected” it is meant that the consensus functional domain target sequence may be selected at the discretion of the practitioner on the basis of known or predicted sequence information, and is not constrained to specific sites recognized by certain site-specific recombinases (e.g., FLP recombinase or CRE recombinase). In some embodiments, the predetermined endogenous DNA target sequence will be other than a naturally occurring germlne DNA sequence (e.g., a transgene, parasitic, mycoplasmal or viral sequence).

In a preferred embodiment, the gene family is the G-protein coupled receptor family, which has only 900 identified members, includes several subfamilies and may include over 13,2000 genes. In a preferred embodiment, the G-protein coupled receptors are from subfamily 1 and are also called R7G proteins. They are an extensive group of receptors which recognize hormones, neurotransmitters, odorants and light and transduce extracellular signals by interaction with guanine (G) nucleotide-binding proteins. The structure of all these receptors is thought to be virtually identical, and they contain seven hydrophobic regions, each of which putatively spans the membrane. The N-terminus is extracellular and is frequently glycosylated, and the C-terminus is cytoplasmic and generally phosphorylated. Three extracellular loops alternate with three cytoplasmic loops to link the seven transmembrane regions. G-protein coupled receptors include, but are not limited to: the class A rhodopsin first subfamily, including amine (acetylcholine (muscarinic), adrenoceptors, domamine, histamine, serotonin, octopamine), peptides (angiotensin, bombesin, bradykinin, C5a anaphylatoxin, Fmet-leu-phe, interleukin-8, chemokine, CCK, endothelin, mealnocortin, neuropeptide Y, neurotensin, opioid, somatostatin, tachykinin, thrombin, vasopressin-like, galanin, proteinase activated), hormone proteins (follicle stimulating hormone, lutropin-choriogonadotropic hormone, thyrotropin), rhodopsin (vertebrate), olfactory (olfactory type 1-11, gustatory), prostanoid (prostaglandin, prostacyclin, thromboxane), nucleotide (adenosine, purinoceptors), cannabis, platelet activating factor, gonadotropin-releasing hormone (gonadotropin releasing hormone, thyrotropin-releasing hormone, growth hormone secretagogue), melatonin, viral proteins, MHC receptor, Mas proto-oncogene, EBV-induced and glucocorticoid induced; the class B secretin second subfamily, including calcitonin, corticotropin releasing factor, gastric inhibitory peptide, glucagon, growth hormone releasing hormone, parathyroid hormone, secretin, vasoactive intestinal polypeptide, and diuretic hormone; the class C metabotropic glutamate third subfamily, including metabrotropic glutamate and extracellular calcium-sensing agents; and the class D pheromone fourth subfamily.

In addition to the first subfamily of G-protein coupled receptors, there is a second subfamily encoding receptors that bind peptide hormones that do not show sequence similarity to the first R7G subfamily. All the characterized receptors in this subfamily are coupled to G-proteins that activate both adenylyl cyclase and the phosphatidylinositol-calcium pathway. However, they are structurally similar; like classical R7G proteins they putatively contain seven transmembrane regions, a glycosylated extracellular N-terminus and a cytoplasmic C-terminus. Known receptors in this subfamily are encoded on multiple exons, and several of these genes are alternatively spliced to yield functionally distinct products. The N-terminuscontains five conserved cysteine residues putatively important in disulfide bonds. Known G-protein coupled receptors in this subfamily are listed above.

In addition to the first and second subfamilies of G-protein coupled receptors, there is a third subfamily encoding receptors that bind glutamate and calcium but do not show sequence similarity to either of the other subfamilies. Structurally, this subfamily has signal sequences, very large hydrophobic extracellular regions of about 540 to 600 amino acids that contain 17 conserved cysteines (putatively involved in disulfides), a region of about 250 residues that appear to contain seven transmembrane domains, and a C-terminal cytoplasmic domain of variable length (50 to 350 residues). Known G-protein coupled receptors of this subfamily are listed above.

In a preferred embodiment, the gene family is the bZIP transcription factor family. This eukaryotic gene family encodes DNA binding transcription factors that contain a basic region that mediates sequence specific DNA binding, and a leucine zipper, required for dimerization. The bZIP family includes, but is not limited to, AP-1, ATF, CREB, CREM, FOS, FRA, GBF, GCN4, HBP, JUN, MET4, OCS1, OP, TAF1, XBP1, and YBBO.

In a preferred embodiment, the gene family is involved in DNA mismatch repair, such as mutL, hexB and PMS1. Members of this family include, but are not limited to, MLH1, PMS1, PMS2, HexB and MulL. The protein consensus sequence is G-F-R-G-E-A-L.

In a preferred embodiment, the gene family is the mutS family, also involved in mismatch repair of DNA, directed to the correction of mismatched base pairs that have been missed by the proofreading element of the DNA polymerase complex. MutS gene family members include, but are not limited to, MSH2, MSH3, MSH6 and MutS.

In a preferred embodiment, the gene family is the recA family. The bacterial recA is essential for homologous recombination and recombinatorial repair of DNA damage. RecA has many activities, including the formation of nucleoprotein filaments, binding to single stranded and double stranded DNA, binding and hydrolyzing ATP, recombinase activity and interaction with lexA causing lexA activation and autocatalytic cleavage. RecA family members include those from E. coli, drosophila, human, lily, etc. specifically including but not limited to, E. coli recA, Rec1, Rec2, Rad51, Rad51B, Rad51C, Rad51D, Rad51E, XRCC2 and DMC1.

In a preferred embodiment, the gene family is the recF family. The prokaryotic recF protein is a single-stranded DNA binding protein which also putatively binds ATP. RecF is involved in DNA metabolism; it is required for recombinatorial DNA repair and for induction of the SOS response. RecF is a protein of about 350 to 370 amino acid residues; there is a conserved ATP-binding site motif ‘A’ in the N-terminal section of the protein as well as two other conserved regions, one located in the central section and the other in the C-terminal section.

In a preferred embodiment, the gene family is the Bcl-2 family. Programmed cell death (PCD), or apoptosis, is induced by events such as growth factor withdrawal and toxins. It is generally controlled by regulators, which have either an inhibitory effect (i.e. anti-apoptotic) or block the protective effect of inhibitors (pro-apoptotic). Many viruses have found a way of countering defensive apoptosis by encoding their own anti-apoptotic genes thereby preventing their target cells from dying too soon.

All proteins belonging to the Bcl-2 family contain at least one of a BH1, BH2, BH3 or BH4 domain. All anti-apoptotic proteins contain BH1 and BH2 domains, some of them contain an additional N-terminal BH4 domain (such as Bcl-2, Bcl-x(L), Bcl-W, etc.), which is generally not found in pro-apoptotic proteins (with the exception of Bcl-x(S). Generally all pro-apoptotic proteins contain a BH3 domain (except for Bad), thought to be crucial for the dimerization of the proteins with other Bcl-2 family members and crucial for their killing activity. In addition, some of the pro-apoptotic proteins contain BH1 and BH2 domains (such as Bax and Bak). The BH3 domain is also present in some anti-apoptosis proteins, such as Bcl-2 and Bcl-x(L). Known Bcl-2 proteins include, but are not limited to, Bcl-2, Bcl-x(L), Bcl-W, Bcl-x(S), Bad, Bax, and Bak.

In a preferred embodiment, the gene family is the site-specific recombinase family. Site-specific recombination plays an important role in DNA rearrangement in prokaryotic organisms. Two types of site-specific recombination are known to occur: a) recombination between inverted repeats resulting in the reversal of a DNA segment; and b) recombination between repeat sequences on two DNA molecules resulting in their cointegration, or between repeats on one DNA molecule resulting the excision of a DNA fragment. Site-specific recombination is characterized by a strand exchange mechanism that requires no DNA synthesis or high energy cofactor; the phosphodiester bond energy is conserved in a phospho-protein linkage during strand cleavage and re-ligation.

Two unrelated families of recombinases are currently known. The first, called the “phage integrase” family, groups a number of bacterial, phage and yeast plasmid enzymes. The second, called the “resolvase” family, groups enzymes which share the following structural characteristics: an N-terminal catalytic and dimerization domain that contains a conserved serine residue involved in the transient covalent attachment to DNA, and a C-terminal helix-turn-helix DNA-binding domain.

In a preferred embodiment, gene family is the single-stranded binding protein family. The E. coli single-stranded binding protein (ssb), also known as the helix-destabilizing protein, is a protein of 177 amino acids. It binds tightly as a homotetramer to a single-stranded DNA ss-DNA) and plays an important role in DNA replication, recombination and repair. Members of the ssb family include, but are not limited to, E. coli ssb and eukaryotic RPA proteins.

In a preferred embodiment, the gene family is the TFIID transcription family. Transcription factor TFIID (or TATA-binding protein, TBP), is a general factor that plays a major role in the activation of eukaryotic genes transcribed by RNA polymerase II. TFIID binds specifically to the TATA box promoter element which lies close to the position of transcription initiation. There is a remarkable degree of sequence conservation of a C-terminal domain of about 180 residues in TFIID from various eukaryotic sources. This region is necessary and sufficient for TATA box binding. The most significant structural feature of this domain is the presence of two conserved repeats of a 77 amino-acid region.

In a preferred embodiment, the gene family is the TGF-β family. Transforming growth factor-β(TGF-β) is a multifunctional protein that controls proliferation, differentiation and other functions in many cell types. TGF-β-1 is a protein of 112 amino acid residues derived by proteolytic cleavage from the C-terminal portion of the precursor protein. Members of the TGF-β family include, but are not limited to, the TGF-1-3 subfamily (including TGF1, TGF2, and TGF3); the BMP3 subfamily (BM3B, BMP3); the BMP5-8 subfamily (BM8A, BMP5, BMP6, BMP7, and BMP8); and the BMP 2 & 4 subfamily (BMP2, BMP4, DECA).

In a preferred embodiment, the gene family is the TNF family. A number of cytokines can be grouped into a family on the basis of amino acid sequence, as well as structural and functional similarities. These include (1) tumor necrosis factor (TNF), also known as cachectin or TNF-α , which is a cytokine with a wide variety of functions. TNF-α can cause cytolysis of certain tumor cell lines; it is involved in the induction of cachexia; it is a potent pyrogen, causing fever by direct action or by stimulation of interleukin-1 secretion; and it can stimulate cell proliferation and induce cell differentiation under certain conditions; (2) lymphotoxin-a (LT-α) and lymphotoxin-β (LT-β), two related cytokines produced by lymphocytes and which are cytotoxic for a wide range of tumor cells in vitro and in vivo; (3) T cell antigen gp39 (CD40L), a cytokine that seems to be important in B-cell development and activation; (4) CD27L, a cytokine that plays a role in T-cell activation; it induces the proliferation of costimulated T cells and enhances the generation of cytolytic T cells; (5) CD30L, a cytokine that induces proliferation of T-cells; (6) FASL, a cytokine involved in cell death; (8) 4-1BBL, an inducible T cell surface molecule that contributes to T-cell stimulation; (9) OX40L, a cytokine that co-stimulates T cell proliferation and cytokine production; and (10), TNF-related apoptosis inducing ligand (TRAIL), a cytokine that induces apoptosis.

In a preferred embodiment, the gene family is the XPA family. Xeroderma pigmentosa (XP) is a human autosomal recessive disease, characterized by a high incidence of sunlight-induced skin cancer. Skin cells associated with this condition are hypersensitive to ultraviolet light, due to defects in the incision step of DNA excision repair. There are a minimum of 7 genetic complementation groups involved in this disorder: XPA to XPG. XPA is the most common form of the disease and is due to defects in a 30 kD nuclear protein called XPA or (XPAC). The sequence of XPA is conserved from higher eukaryotes to yeast (gene RAD14). XPA is a hydrophilic protein of 247 to 296 amino acid residues that has a C4-type zinc finger motif in its central section.

In a preferred embodiment, the gene family is the XPG family. The defect in XPG can be corrected by a 133 kD nuclear protein called XPG (or XPGC). Members of the XPG family include, but are not limited to, FEN1, XPG, RAD2, EXO1, and DIN7.

The invention further comprises adding at least one recombinase coated single stranded nucleic acid probe to the target sequence. By “recombinase coated” is meant at least one recombinase is non-covalently associated with the single stranded nucleic acid probe. By “recombinase” herein is meant a protein that, when included with an exogenous single stranded nucleic acid probe, provide a measurable increase in the recombination frequency and/or localization frequency between the single stranded nucleic acid probe and an endogenous predetermined DNA sequence. Thus, in a preferred embodiment, increases in recombination frequency from the normal range of 10⁻⁸ to 10⁻⁴, to 10⁻⁴ to 10¹, preferably 10⁻³ to 10¹, and most preferably 10⁻² to 10⁻⁴ , may be achieved.

In the present invention, recombinase refers to a family of RecA-like recombination proteins all having essentially all or most of the same functions, particularly: (i) the recombinase protein's ability to properly bind to and position single stranded nucleic acid probe s on their homologous targets and (ii) the ability of recombinase protein/single stranded nucleic acid probe complexes to efficiently find and bind to complementary endogenous sequences. The best characterized recA protein is from E. coli, in addition to the wild-type protein a number of mutant recA proteins have been identified (e.g., recA803; see Madiraju et al., PNAS USA 85(18):6592 (1988); Madiraju et al, Biochem. 31:10529 (1992); Lavery et al., J. Biol. Chem. 267:20648 (1992)). Further, many organisms have recA-like recombinases with strand-transfer activities (e.g., Fugisawa et al., (1985) Nucl. Acids Res. 13: 7473; Hsieh et al., (1986) Cell 44:885; Hsieh et al., (1989) J. Biol. Chem. 264:5089; Fishel et al., (1988) Proc. Nati. Acad. Sci. (USA) 85:3683; Cassuto et al., (1987) Mol. Gen. Genet. 208:10; Ganea et al., (1987) Mol. Cell Biol. 7-3124; Moore et al., (1990) J. Biol. Chem. 19:11108; Keene et al., (1984) Nucl. Acids Res. 12: 3057; Kimeic, (1984) Cold Spring Harbor Symp. 48:675; Kmeic, (1986) Cell 44:545; Kolodner et al., (1987) Proc. Natl. Acad. Sci. USA 84:5560; Sugino et al., (1985) Proc. Natl. Acad. Sci. USA 85:3683; Halbrook et al., (1989) J. Biol. Chem. 264:21403; Eisen et al., (1988) Proc. Natl. Acad. Sci. USA 85:7481; McCarthy et al., (1988) Proc. Nati. Acad. Sci. USA 85:5854; Lowenhaupt et al., (1989) J. Biol. Chem. 264:20568, which are incorporated herein by reference. Examples of such recombinase proteins include, for example but not limited to: recA, recA803, uvsX, and other recA mutants and recA-like recombinases (Roca, A. I. (1990) Crit. Rev. Biochem. Molec. Biol. 25:415), sep1 (Kolodner et al. (1987) Proc. Natl. Acad. Sci. (U.S.A.) 84:5560; Tishkoff et al. Molec. Cell. Biol. 11:2593), RuvC (Dunderdale et al. (1991) Nature 354:506), DST2, KEM1, XRN1 (Dykstra et al. (1991) Molec. Cell. Biol. 11:2583), STPα/DST1 (Clark et al. (1991) Molec. Cell. Biol. 11:2576), HPP-1 (Moore et al. (1991) Proc. Nati. Acad. Sci. (U.S.A.) 88:9067), other target recombinases (Bishop et al. (1992) Cell 69:439; Shinohara et al. (1992) Cell 69:457); incorporated herein by reference. RecA may be purified from E. coli strains, such as E. coli strains JC12772 and JC15369 (available from A. J. Clark and M. Madiraju, University of California-Berkeley, or purchased commercially). These strains contain the recA coding sequences on a “runaway” replicating plasmid vector present at a high copy numbers per cell. The recA803 protein is a high-activity mutant of wild-type recA. The art teaches several examples of recombinase proteins, for example, from Drosophila, yeast, plant, human, and non-human mammalian cells, including proteins with biological properties similar to recA (i.e., recA-like recombinases), such as Rad51, Rad57, dmel from mammals and yeast, and Pk-rec (see Rashid et al., Nucleic Acid Res. 25(4):719 (1997), hereby incorporated by reference). In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli brecA803 mutant with enhanced recombinase activity.

In a preferred embodiment, recA or rad51 is used. For example, recA protein is typically obtained from bacterial strains that overproduce the protein: wild-type E. coli recA protein and mutant recA803 protein may be purified from such strains. Alternatively, recA protein can also be purchased from, for example, Pharmacia (Piscataway, N.J.) or Boehringer Mannheim (Indianapolis, Ind.).

RecA proteins, and its homologs, form a nucleoprotein filament when it coats a single-stranded DNA. In this nucleoprotein filament, one monomer of recA protein is bound to about 3 nucleotides. This property of recA to coat single-stranded DNA is essentially sequence independent, although particular sequences favor initial loading of recA onto a polynucleotide (e.g., nucleation sequences). The nucleoprotein filament(s) can be formed on essentially any DNA molecule and can be formed in cells (e.g., mammalian cells), forming complexes with both single-stranded and double-stranded DNA, although the loading conditions for dsDNA are somewhat different than for ssDNA.

In a preferred embodiment, the single stranded nucleic acid probes are coated with recombinase prior to introduction to the target sequence. The conditions used to coat single stranded nucleic acid probes with recombinases such as recA protein and ATPyS have been described in commonly assigned U.S. Ser. No. 07/910,791, filed Jul. 9, 1992; U.S. Ser.No. 07/755,462, filed Sep. 4, 1991, and PCT U.S. Ser. No. 07/520,321, filed May 7, 1990, and PCT US Ser. No. 98/05223, each incorporated herein by reference. The procedures below are directed to the use of E. coli recA, although as will be appreciated by those in the art, other recombinases may be used as well. Single stranded nucleic acid probes can be coated using GTPyS, mixes of ATPyS with rATP, rGTP and/or dATP, or dATP or rATP alone in the presence of an rATP generating system (Boehringer Mannheim). Various mixtures of GTPyS, ATPyS, ATP, ADP, dATP and/or rATP or other nucleosides may be used, particularly preferred are mixes of ATPyS and ATP or ATPyS and ADP.

RecA protein coating of single stranded nucleic acid probe s is typically carried out as described in U.S. Ser. No. 07/910,791, filed Jul. 9, 1992 and U.S. Ser. No. 07/755,462, filed Sep. 4, 1991, and PCT US Ser. No. 98/05223, which are incorporated herein by reference. Briefly, the single stranded nucleic acid probe, is denatured by heating in an aqueous solution at 95-100° C. for five minutes, then placed in an ice bath for 20 seconds to about one minute followed by centrifugation at 0° C. for approximately 20 sec, before use. When denatured single stranded nucleic acid probes are not placed in a freezer at −20° C. they are usually immediately added to standard recA coating reaction buffer containing ATPyS, at room temperature, and to this is added the recA protein. Alternatively, recA protein may be included with the buffer components and ATPyS before the polynucleotides are added.

RecA coating of single stranded nucleic acid probe(s) is initiated by incubating polynucleotide-recA mixtures at 37° C. for 10-15 min. RecA protein concentration tested during reaction with polynucleotide varies depending upon polynucleotide size and the amount of added polynucleotide, and the ratio of recA molecule:nucleotide preferably ranges between about 3:1 and 1:3. When single-stranded polynucleotides are recA coated independently of their homologous polynucleotide strands, the mM and pM concentrations of ATPyS and recA, respectively, can be reduced to one-half those used with double-stranded single stranded nucleic acid probes (i.e., recA and ATPyS concentration ratios are usually kept constant at a specific concentration of individual polynucleotide strand, depending on whether a single- or double-stranded polynucleotide is used).

RecA protein coating of single stranded nucleic acid probes is normally carried out in a standard 1X RecA coating reaction buffer. 10X RecA reaction buffer (i.e., 10x AC buffer) consists of: 100 mM Tris acetate (pH 7.5 at 37° C.), 20 mM magnesium acetate, 500 mM sodium acetate, 10 mM DTT, and 50% glycerol). All of the single stranded nucleic acid probes, whether double-stranded or single-stranded, typically are denatured before use by heating to 95-100° C. for five minutes, placed on ice for one minute, and subjected to centrifugation (10,000 rpm) at 0° C. for approximately 20 seconds (e.g., in a Tomy centrifuge). Denatured single stranded nucleic acid probes usually are added immediately to room temperature RecA coating reaction buffer mixed with ATPyS and diluted with double-distilled H₂O as necessary. A reaction mixture typically contains the following components: (i) 0.2-4.8 mM ATPyS; and (ii) between 1-100 nglpl of single stranded nucleic acid probe. To this mixture is added about 1-20 μ of recA protein per 10-100 μ of reaction mixture, usually at about 2-10 mg/ml (purchased from Pharmacia or purified), and is rapidly added and mixed. The final reaction volume-for RecA coating of single stranded nucleic acid probe is usually in the range of about 10-500 μl. RecA coating of single stranded nucleic acid probe is usually initiated by incubating single stranded nucleic acid probe-RecA mixtures at 37° C. for about 10-15 min.

RecA protein concentrations in coating reactions varies depending upon single stranded nucleic acid probe size and the amount of added single stranded nucleic acid probe: recA protein concentrations are typically in the range of 5 to 50 μM. When single-stranded single stranded nucleic acid probes are coated with recA, independently of their complementary strands, the concentrations of ATPYS and recA protein may optionally be reduced to about one-half of the concentrations used when the complementary strands of-single stranded nucleic acid probes of the same length are used: that is, the recA protein and ATPyS concentration ratios are generally kept constant for a given concentration of individual polynucleotide strands.

The coating of single stranded nucleic acid probes with recA protein can be evaluated in a number of ways. First, protein binding to DNA can be examined using band-shift gel assays (McEntee et al., (1981) J. Biol. Chem. 256:8835). Labeled polynucleotides can be coated with recA protein in the presence of ATPyS and the products of the coating reactions may be separated by agarose gel electrophoresis. Following incubation of recA protein with denatured duplex DNAs the recA protein effectively coats single-stranded single stranded nucleic acid probes derived from denaturing a duplex DNA. As the ratio of recA protein monomers to nucleotides in the single stranded nucleic acid probe increases from 0, 1:27, 1:2.7 to 3.7:1 for 121-mer and 0, 1:22, 1:2.2 to 4.5:1 for 159-mer, single stranded nucleic acid probe's electrophoretic mobility decreases, i.e., is retarded, due to recA-binding to the single stranded nucleic acid probe. Retardation of the coated polynucleotide's mobility reflects the saturation of single stranded nucleic acid probe with recA protein. An excess of recA monomers to DNA nucleotides is required for efficient recA coating of short single stranded nucleic acid probes (Leahy et al., (1986) J. Biol. Chem. 261:954).

A second method for evaluating protein binding to DNA is in the use of nitrocellulose fiber binding assays (Leahy et al., (1986) J. Biol. Chem. 261:6954; Woodbury, et al., (1983) Biochemistry 22(20):4730-4737. The nitrocellulose filter binding method is particularly useful in determining the dissociation-rates for protein:DNA complexes using labeled DNA. In the filter binding assay, DNA:protein complexes are retained on a filter while free DNA passes through the filter. This assay method is more quantitative for dissociation-rate determinations because the separation of DNA:protein complexes from free single stranded nucleic acid probe is very rapid.

Alternatively, recombinase protein(s) (prokaryotic, eukaryotic or endogenous to the target cell) may be exogenously induced or administered to a target cell simultaneously or contemporaneously (i.e., within about a few hours) with the single stranded nucleic acid probe(s). Such administration is typically done by micro-injection, although electroporation, lipofection, and other transfection methods known in the art may also be used. Alternatively, recombinase-proteins may be produced in vivo. For example, they may be produced from a homologous or heterologous expression cassette in a transfected cell or targeted cell, such as a transgenic totipotent cell (e.g. a fertilized zygote) or an embryonal stem cell (e.g., a murine ES cell such as AB-1) used to generate a transgenic non-human animal line or a somatic cell or a pluripotent hematopoietic stem cell for reconstituting all or part of a particular stem cell population (e.g. hematopoietic) of an individual. Conveniently, a heterologous expression cassette includes a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible drug inducible, or a tetracyclin inducible, or other modulatable promoter construct so that expression of at least one species of recombinase protein from the cassette can by modulated for transiently producing recombinase(s) in vivo simultaneous or contemporaneous with introduction of a single stranded nucleic acid probe into the cell. When a hormone-inducible promoter-enhancer combination is used, the cell must have the required hormone receptor present, either naturally or as a consequence of expression a co-transfected expression vector encoding such receptor. Alternatively, the recombinase may be endogenous and produced in high levels. In this embodiment, preferably in eukaryotic target cells such as tumor cells, the target cells produce an elevated level of recombinase. In other embodiments the level of recombinase may be induced by DNA damaging agents, such as mitomycin C, UV or y-irradiation. Alternatively, recombinase levels may be elevated by transfection of a plasmid encoding the recombinase gene into the cell.

In general, the single stranded nucleic acid probes may comprise any number of structures, as long as the changes do not substantially effect the functional ability of the single stranded nucleic acid probe to result in homologous recombination. For example, recombinase coating of alternate structures should still be able to occur.

In addition to recombinases, the single stranded nucleic acid probes of the invention may comprise additional components, such as cell-uptake components, chemical substituents, purification tags, etc. Analog probes may also comprise these additional components.

In a preferred embodiment, at least one of the probes of the invention comprises at least one celluptake component. As used herein, the term “cell-uptake component” refers to an agent which, when bound, either directly or indirectly, to a probe, enhances the intracellular uptake of the probe into at least one cell type (e.g., hepatocytes). A probe of the invention may optionally be conjugated, typically by covalently or preferably noncovalent binding, to a cell-uptake component. Various methods have been described in the art for targeting DNA to specific cell types. A probe of the invention can be conjugated to essentially any of several cell-uptake components known in the art. For targeting to hepatocytes, a probe can be conjugated to an asialoorosomucoid (ASOR)-poly-L-lysine conjugate by methods described in the art and incorporated herein by reference (Wu GY and Wu CH (1987) J. Biol. Chem. 262:4429; Wu GY and Wu CH (1988) Biochemistry 27:887; Wu GY and Wu CH (1988) J. Biol. Chem. 263:14621; Wu GY and Wu CH (1992) J. Biol. Chem. 267:12436; Wu et al. (1991) J. Biol. Chem. 266:14338; and Wilson et al. (1992) J. Biol. Chem. 267: 963, WO92/06180; WO92/05250; and WO91/17761, which are incorporated herein by reference).

Alternatively, a cell-uptake component may be formed by incubating the probe with at least one lipid species and at least one protein species to form protein-lipid-polynucleotide complexes consisting essentially of the single stranded nucleic acid probe or analog probe and the lipid-protein cell-uptake component. Lipid vesicles made according to Felgner (W Ser. No. 091/17424, incorporated herein by reference) and/or cationic lipidization (WO Ser. No. 91/16024, incorporated herein by reference) or other forms for polynucleotide administration (EP 465,529, incorporated herein by reference) may also be employed as cell-uptake components. Nucleases may also be used.

In addition to cell-uptake components, targeting components such as nuclear localization signals may be used, as is known in the art. See for example Kido et al., Exper. Cell Res. 198:107-114 (1992), hereby expressly incorporated by reference.

Typically, in this embodiment a single stranded nucleic acid probe of the invention is coated with at least one recombinase and is conjugated to a cell-uptake component, and the resulting cell targeting complex is contacted with a target cell under uptake conditions (e.g., physiological conditions) so that the single stranded nucleic acid probe and the recombinase(s) are internalized in the target cell. A single stranded nucleic acid probe may be contacted simultaneously or sequentially with a cell-uptake component and also with a recombinase; preferably the single stranded nucleic acid probe is contacted first with a recombinase, or with a mixture comprising both a cell-uptake component and a recombinase under conditions whereby, on average, at least about one molecule of recombinase is noncovalently attached per single stranded nucleic acid probe molecule and at least about one cell-uptake component also is noncovalently attached. Most preferably, coating of both recombinase and cell-uptake component saturates essentially all of the available binding sites on the single stranded nucleic acid probe. A single stranded nucleic acid probe may be preferentially coated with a cell-uptake component so that the resultant targeting complex comprises, on a molar basis, more cell-uptake component than recombinase(s). Alternatively, a single stranded nucleic acid probe may be preferentially coated with recombinase(s) so that the resultant targeting complex comprises, on a molar basis, more recombinase(s) than cell-uptake component.

Cell-uptake components are included with recombinase-coated single stranded nucleic acid probes or analog probes of the invention to enhance the uptake of the recombinase-coated single stranded nucleic acid probe(s) or analog probe into cells, particularly for in vivo gene targeting applications, such as gene therapy to treat genetic diseases, including neoplasia, and targeted homologous recombination to treat viral infections wherein a viral sequence (e.g., an integrated hepatitis B virus (HBV) genome or genome fragment) may be targeted by homologous sequence targeting and inactivated. Alternatively, a single stranded nucleic acid probe or analog probe may be coated with the cell-uptake component and targeted to cells with a contemporaneous or simultaneous administration of a recombinase (e.g., liposomes or immunoliposomes containing a recombinase, a viral-based vector encoding and expressing a recombinase).

In addition to cellular uptake components, at least one of the probes may include chemical substituents. Probes that have been modified with appended chemical substituents may be introduced along with recombinase (e.g., recA) into a metabolically active target cell to homologously pair with a predetermined endogenous DNA target sequence in the cell. In a preferred embodiment, the probes are derivatized, and additional chemical substituents are attached, either during or after polynucleotide synthesis, respectively, and are thus localized to a specific nucleic acid target sequence where they produce an alteration or chemical modification to a local nucleic acid sequence. Preferred attached chemical substituents include, but are not limited to: cross-linking agents (see Podyminogin et al., Biochem. 34:13098 (1995) and 35:7267 (1996), both of which are hereby incorporated by reference), nucleic acid cleavage agents, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, labels, base-modification agents, agents which normally bind to nucleic acids such as labels, etc. (see for example Afonina et al., PNAS USA 93:3199 (1996), incorporated herein by reference) immunoglobulin chains, and oligonucleotides. Iron/EDTA chelates are particularly preferred chemical substituents where local cleavage of a DNA sequence is desired (Hertzberg et al. (1982) J. Am. Chem. Soc. 104:313; Hertzberg and Dervan (1984) Biochemistry 23:3934; Taylor et al. (1984) Tetrahedron 40:457; Dervan, PB (1986) Science 232:464, which are incorporated herein by reference). Further preferred are groups that prevent hybridization of the complementary single stranded nucleic acids to each other but not to unmodified nucleic acids; see for example Kutryavin et al., Biochem. 35:11170 (1996) and Woo et al., Nucleic Acid. Res. 24(13):2470 (1996), both of which are incorporated by reference. 2′-0 methyl groups are also preferred; see Cole-Strauss et al., Science 273:1386 (1996); Yoon et al., PNAS 93:2071 (1996)). Additional preferred chemical substituents include labeling moieties, including fluorescent labels. Preferred attachment chemistries include: direct linkage, e.g., via an appended reactive amino group (Corey and Schultz (1988) Science 238:1401, which is incorporated herein by reference) and other direct linkage chemistries, although streptavidin/biotin and digoxigenin/antidigoxigenin antibody linkage methods may also be used. Methods for linking chemical substituents are provided in **U.S. Pat. Nos. 5,135,720, 5,093,245, and 5,055,556, which are incorporated herein by reference. Other linkage chemistries may be used at the discretion of the practitioner.

In a preferred embodiment, at least one of the single stranded nucleic acid probes or analog probes comprises at least one purification tag or capture moiety, some of which are discussed above as chemical substituents, for example biotin, digoxigenin, psoralen, etc. Alternatively, the probe could be directly attached to beads with the targeting reaction performed on a solid phase support.

In a preferred embodiment, at least one of the probes of the invention (including the single stranded nucleic acid or the analog probes) may further comprise a separation moiety. By “separation moiety” or “purification moiety” or grammatical equivalents herein is meant a moiety which may be used to purify or isolate the nucleic acids, including the nucleic acid analog probes, the single stranded nucleic acid probe:target sequence complex, or the target sequence. As will be appreciated by those in the art, the separation moieties may comprise any number of different entities, including, but not limited to, haptens such as chemical moieties, epitope tags, binding partners, or unique nucleic acid sequences; basically anything that can be used to isolate or separate a nucleic acid analog probe:target sequence complex from the rest of the nucleic acids present.

For example, in a preferred embodiment, the separation moiety is a binding partner pair, such as biotin, such that biotinylated targeting probes are made, and streptavidin or avidin columns or beads plates (particularly magnetic beads as described herein) can be used to isolate the targeting probe:target sequence complex.

Alternatively, the rescue sequence may be a unique oligonucleotide sequence which serves as a probe target site to allow the quick and easy isolation of the retroviral construct, via PCR, related techniques, or hybridization.

In a preferred embodiment, the separation moiety is an epitope tag for detection, immunoprecipitation or FACS (fluorescence-activated cell sorting). Suitable epitope tags include myc (for use with the commercially available 9E10 antibody), the BSP biotinylation target sequence of the bacterial enzyme BirA, flu tags, lacZ, and GST.

Alternatively, the separation moiety may be a separation sequence that is a unique oligonucleotide sequence which serves as a probe target site to allow the quick and easy isolation of the complex; for example using an affinity-type column.

In a preferred embodiment, the separation sequence is a peptide sequence that includes purification sequences such as the His₆ tag for use with Ni affinity columns.

Accordingly, the present invention provides compositions comprising one or more analog probes and one or more single stranded nucleic acid probes coated with recombinase.

RecA-protein-mediated D-loops may be formed between one single stranded nucleic acid probe and a nucleic acid target sequence. Internally located double stranded nucleic acid target sequences on relaxed linear DNA targets hybridized by single stranded nucleic acid probes produce single D-loops comprising a three-stranded complex. The addition of a second complementary single stranded nucleic acid probe to the three-strand-containing single-D-loop stabilizes the deproteinized hybrid joint molecules by allowing W-C base pairing of the probe with the displaced target DNA strand. The addition of a second RecA-coated complementary single stranded nucleic acid probe strand to the three-strand containing single D-loop stabilizes deproteinized hybrid joints located away from the free ends of the duplex target DNA (Sena & Zarling, Nature Genetics 3:365 (1993); Revet et al. J. Mol. Biol. 232:779 (1993); Jayasena and Johnston, J. Mol. Bio. 230:1015 (1993)). The resulting four-stranded structure, named a double D-loop by analogy with the three-stranded single D-loop hybrid has been shown to be stable in the absence of RecA protein. This stability likely occurs because the restoration of W-C base pairing in the parental duplex would require disruption of two W-C base pairs in the double-D-loop (one W-C pair in each heteroduplex D-loop). Since each base-pairing in the reverse transition (double-D-loop to duplex) is less favorable by the energy of one W-C base pairs, the pair of single stranded nucleic acid probes are thus kinetically trapped in duplex DNA targets in stable hybrid structures. The stability of the double-D loop joint molecule within internally located probe:target hybrids is an intermediate stage prior to the progression of the homologous recombination reaction to the strand exchange phase. The double D-loop permits isolation of stable multistranded DNA recombination intermediates.

Although nucleic acid target sequence recombination may be mediated by the addition of recombinase to single stranded nucleic acid probe(s), the yields of these recombination events may be less than 100%, especially after hybrid deproteinization. An object of this invention is to improve recombination frequency, speed and/or efficiency using analog probes.

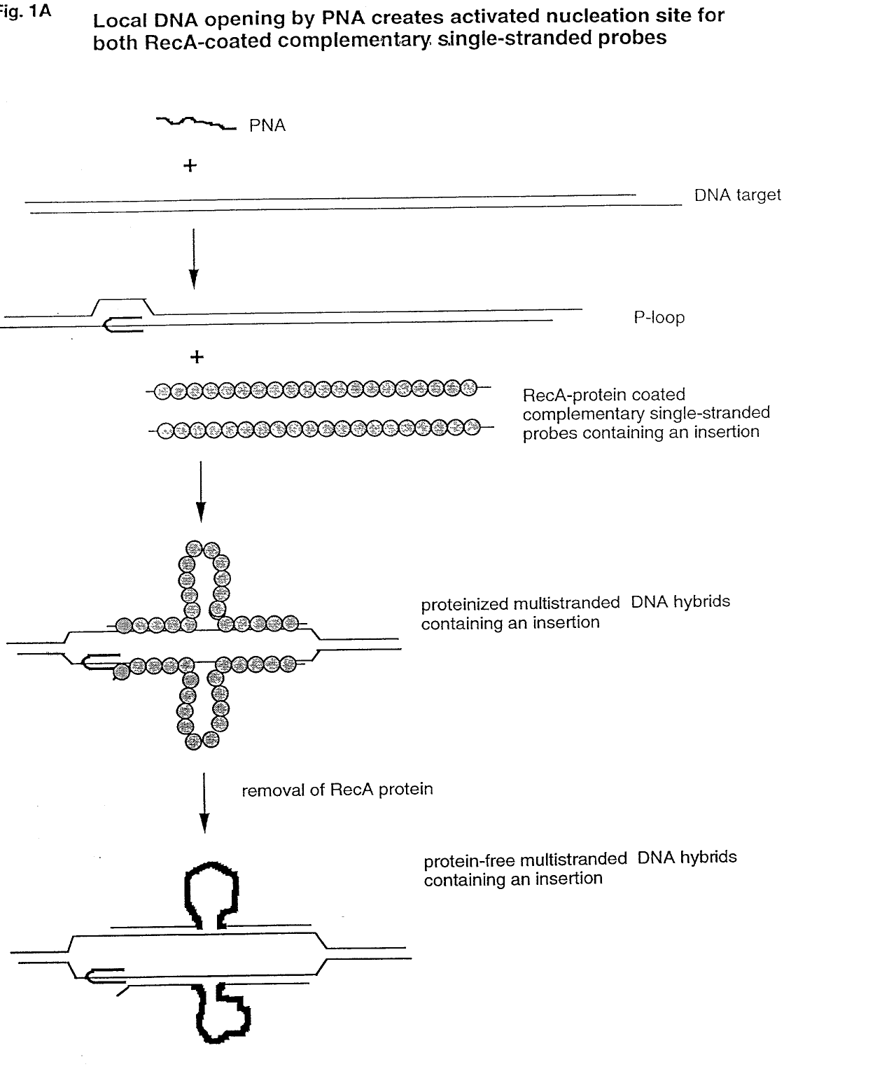

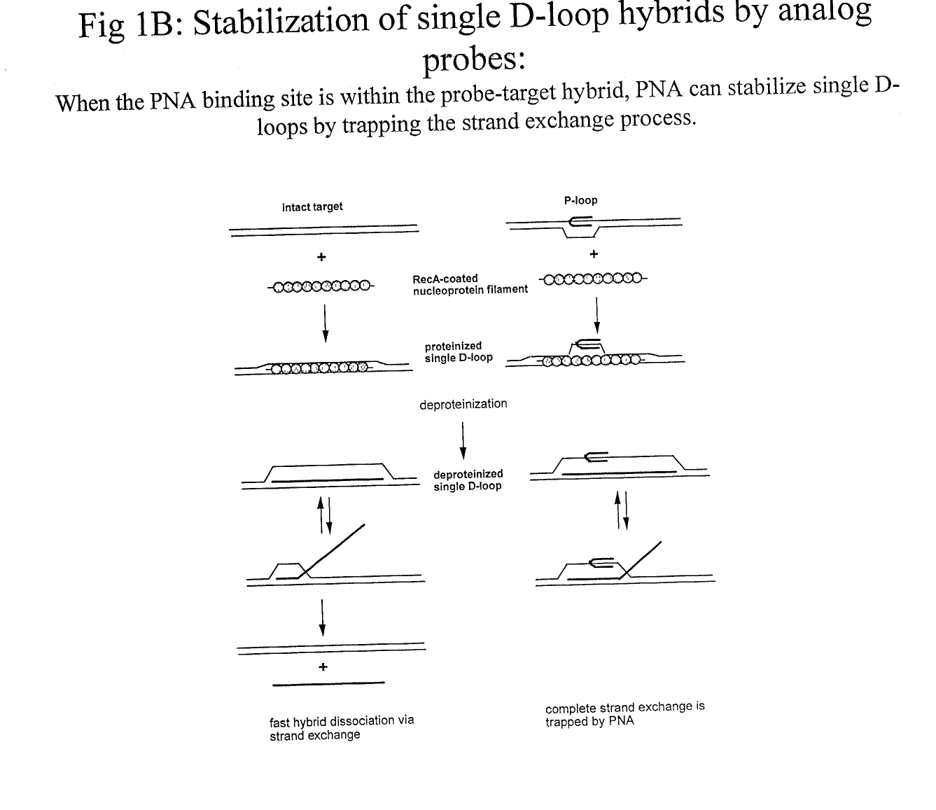

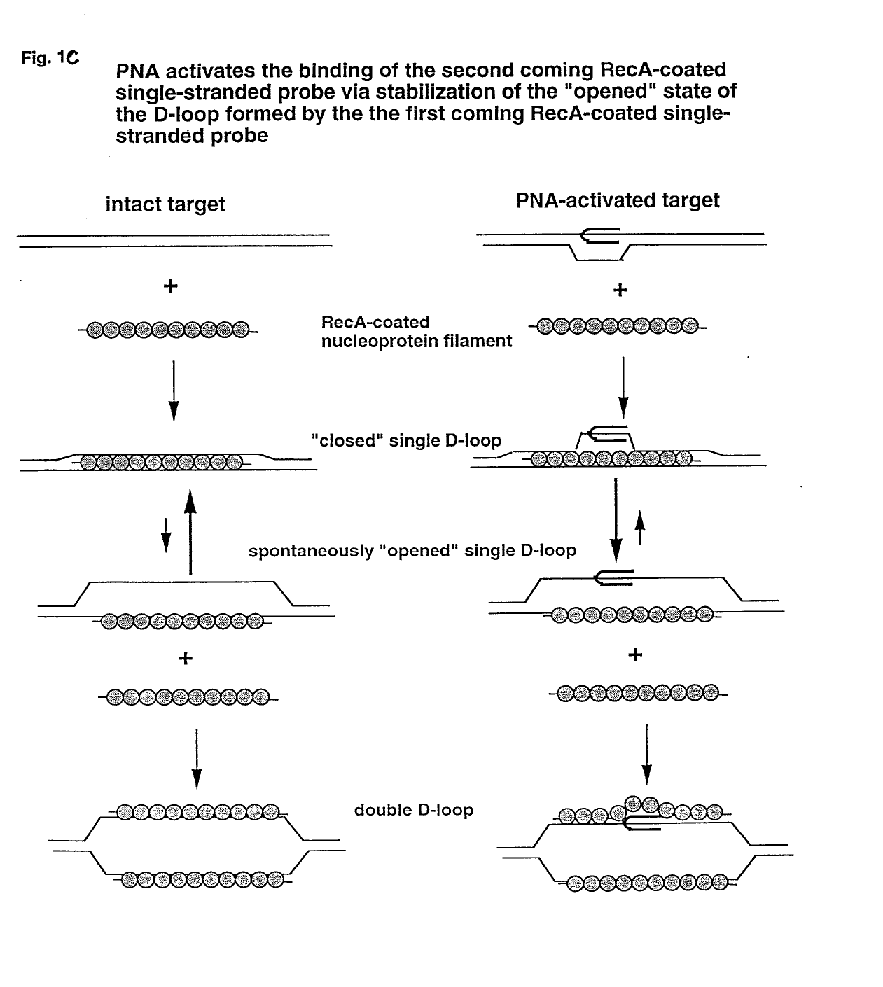

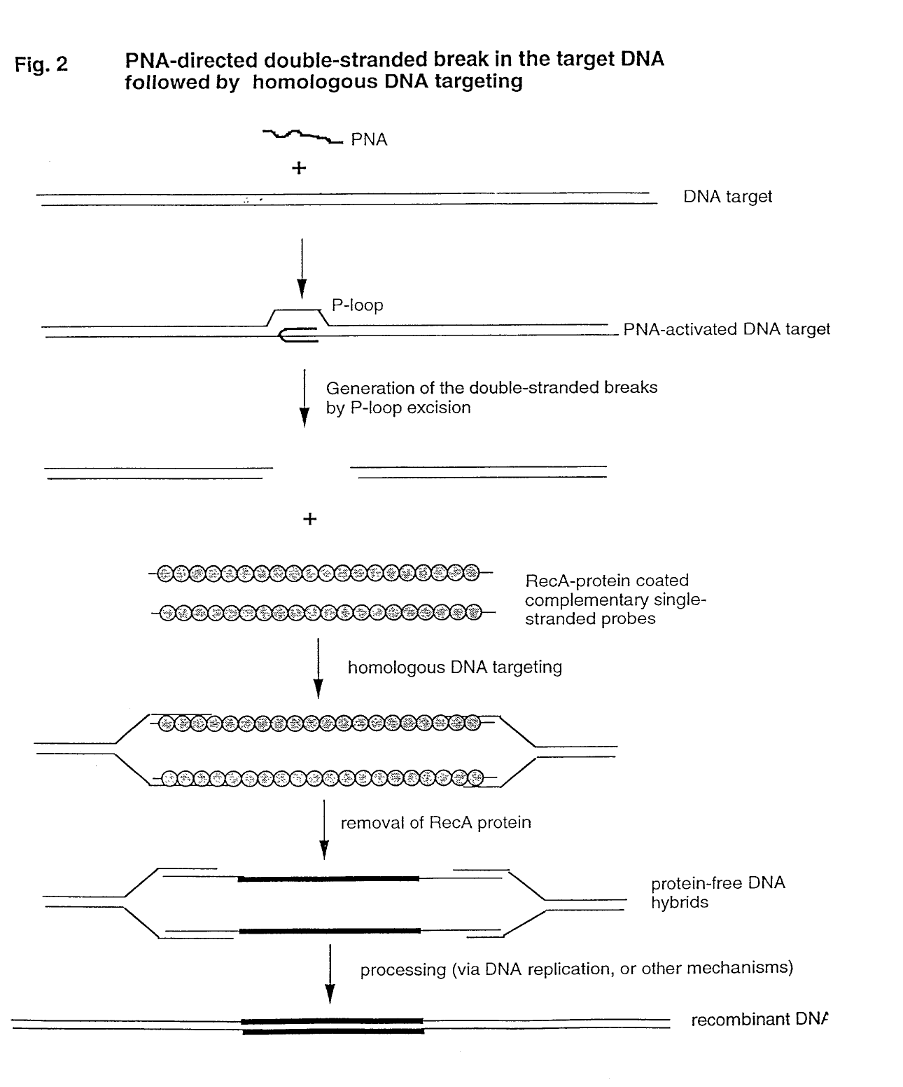

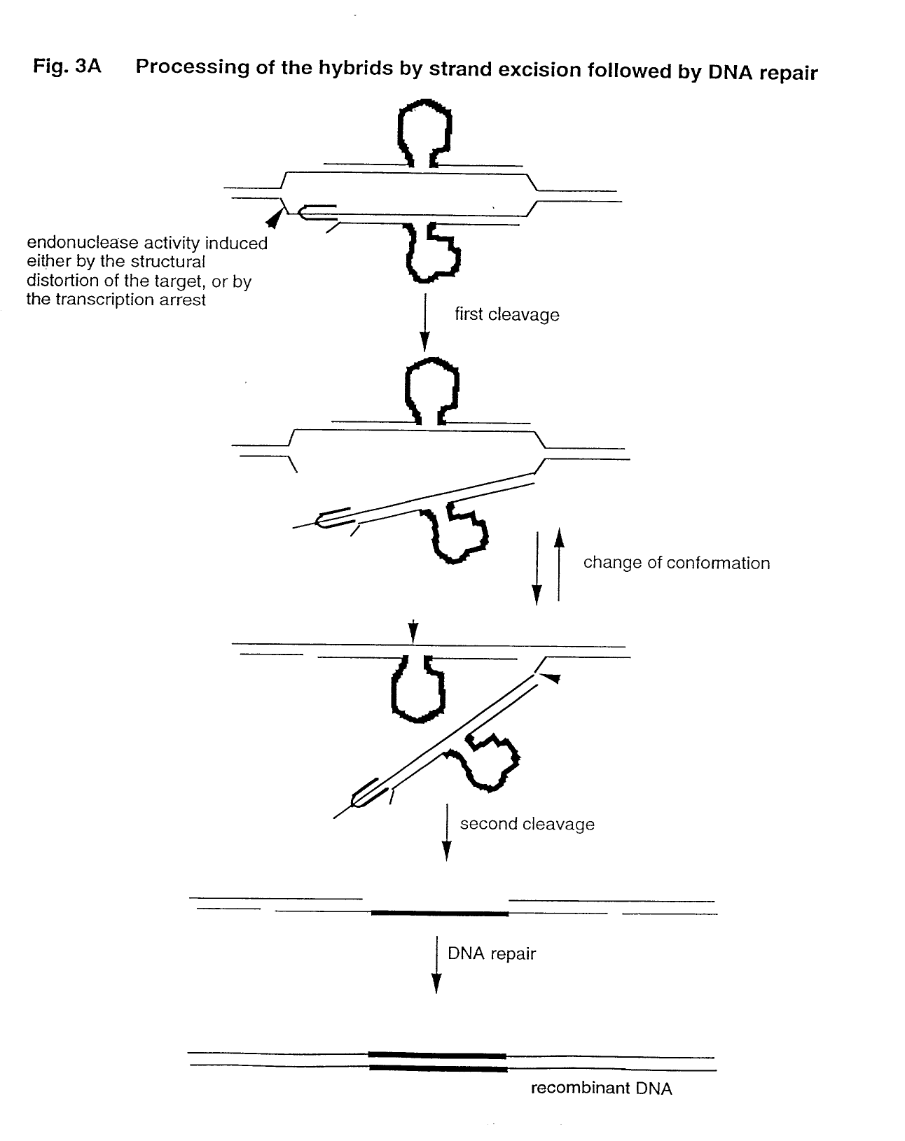

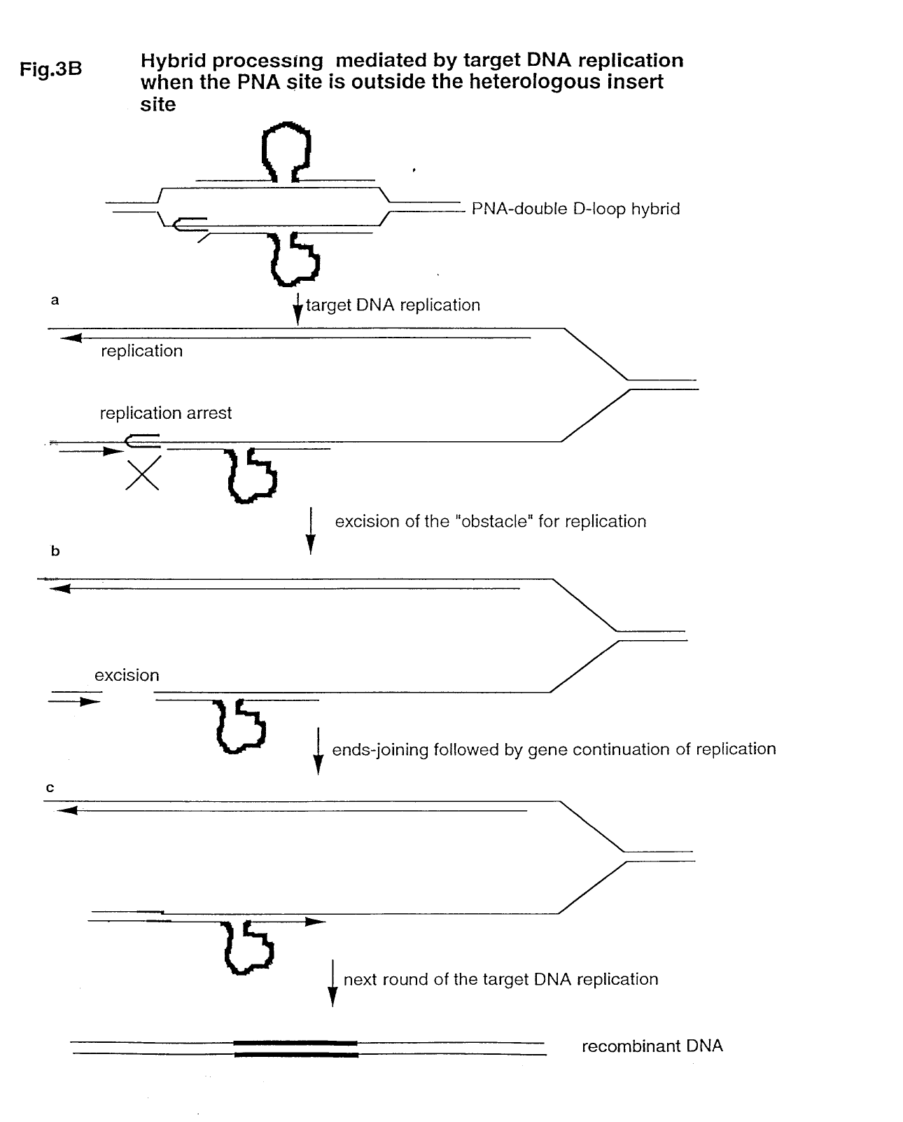

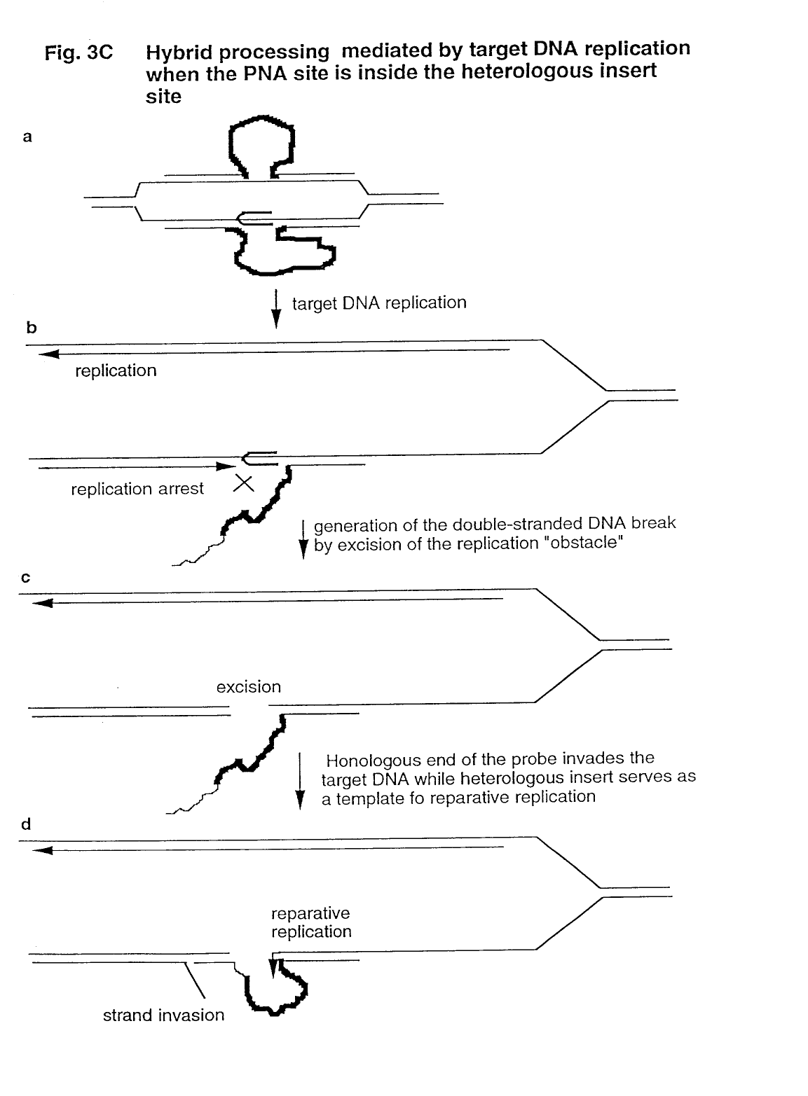

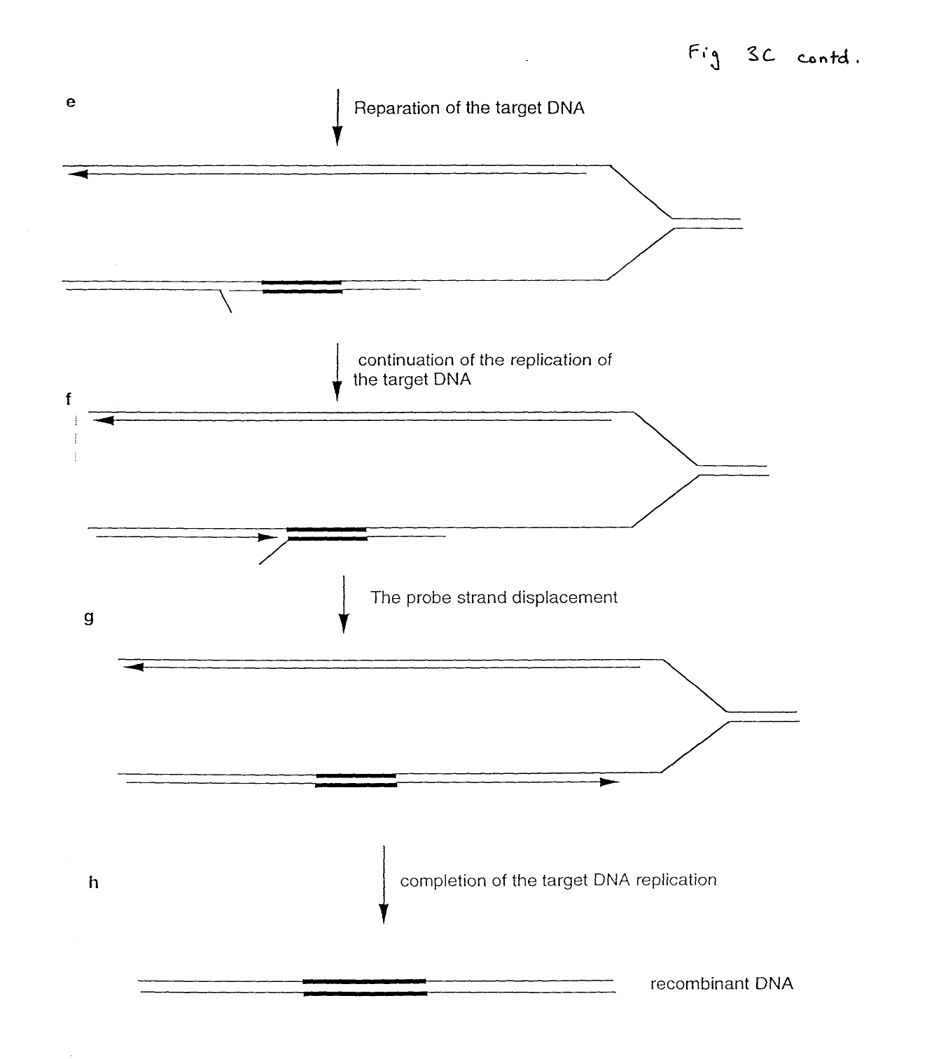

In a preferred embodiment, analog probes improve the kinetics of recA-mediated strand exchange by providing a local opening in the target nucleic acid sequence. The open regions provided by analog-nucleic acid target sequence hybridization facilitates the recombinase driven strand-exchange by initiating base-pair opening, as shown in FIGS. 1A, 1B, 1C. In a similar way, one analog probe accelerates the binding of the another analog probe into the adjacent site (Kurakin et al., 1998 in Chem Biol, 5, 81-89), and the open flanks of cruciform structures accelerate D-loop formation in superhelical DNA (lyeretal., 1995, J. Biol. Chem., 27, 14712-14717).

In a preferred embodiment, a PNA analog probe enhances double D-loop formation by shifting the equilibrium of the D-loop conformation to an “open” conformation (FIG. 1C). In this reaction, the RecA coated single stranded nucleic acid probe and the double stranded nucleic acid target sequence remain bound within a three stranded complex (for example, see Reddy et al, 1994, Biochemistry, 33,11486-11492; Malkov, 2000, J Mol Biol, 299, 629-640). Due to thermal fluctuations, the complex can reversibly from an “open” single D-loop (Reddy, 1994, Biochemistry, 33,11486-11492), which hybridizes to a second RecA coated single stranded nucleic acid probe much more readily than the “closed” single D-loop. The PNA analog probe distorts the “closed” hybrid complex, thus shifting the equilibrium towards the “open” hybrid complex and, consequently, facilitating the hybridization of the second RecA coated single stranded nucleic acid probe.

In a preferred embodiment, the analog probe facilitates single stranded nucleic acid hybridization by binding to a single strand of the nucleic acid target sequence. The analog probe may bind to some portion of the target sequence or within the vicinity of the target sequence.

In a preferred embodiment, the analog probe sequence facilitates single stranded nucleic acid hybridization by simultaneously binding to both strands of the double stranded target sequence. The analog probe may bind to some portion of the target sequence or within the vicinity of the target sequence.

Once made, the compositions of the invention find use in a number of applications in vivo or in vitro, including site directed modification of endogenous sequences within any target cell, the creation of transgenic plants and animals, and the use of the compositions to do site-directed mutagenesis or modifications of target sequences, and cloning of target sequences.

Accordingly, the compositions of the invention may be added to cells. Once the analog probe and recombinase coated single stranded nucleic acid probe compositions are formulated, they are introduced or administered into target cells. The administration is typically done as is known for the administration of nucleic acids into cells, and, as those skilled in the art will appreciate, the methods may depend on the choice of the target cell. Suitable methods include, but are not limited to, microinjection, electroporation, lipofection, etc. By “target cells” herein is meant prokaryotic or eukaryotic cells. Suitable prokaryotic cells include, but are not limited to, bacteria such as E. coli,Bacillus species, and the extremophile bacteria such as thermophiles, etc. Suitable eukaryotic cells include, but are not limited to, fungi such as yeast and filamentous fungi, including species of Aspergillus, Trichoderma, and Neurospora; plant cells including those of corn, sorghum, tobacco, canola, soybean, cofton, tomato, etc.; and animal cells, including fish, birds and mammals. Suitable fish cells include, but are not limited to, those from species of salmon, trout, tulapia and zebrafish. Suitable bird cells include, but are not limited to, those of chickens, turkeys, ducks, quail, pheasants, ostrich and other game birds. Suitable mammalian cells include, but are not limited to, cells from horses, cows, buffalo, deer, sheep, rabbits, rodents such as mice, rats, hamsters and guinea pigs, goats, pigs, primates, marine mammals including dolphins and whales, as well as cell lines, such as human cell lines of any tissue or stem cell type, and stem cells, including pluripotent and non-pluripotent, and non-human zygotes. Particular human cells including, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, mouse La, HT1080, C127, Rat2, CV-1, NIH3T3 cells, CHO, COS, 293 cells, etc. See the ATCC cell line catalog, hereby expressly incorporated by reference.

The vectors containing the DNA segments of interest can be transferred into the host cell by well-known methods, depending on the type of cellular host. For example, micro-injection is commonly utilized for target cells, although calcium phosphate treatment, electroporation, lipofection, biolistics or viral-based transfection also may be used. Other methods used to transform mammalian cells include the use of Polybrene, protoplast fusion, and others (see, generally, Sambrook et al. Molecular Cloning: A Laboratory Manual, 2d ed., 1989, Cold Spring Harbor Laboratory Press, Cold Spring Harbor, N.Y., which is incorporated herein by reference). Direct injection of DNA and/or recombinase-coated single stranded nucleic acid probes into target cells, such as skeletal or muscle cells also may be used (Wolff et al. (1990) Science 247:1465, which is incorporated herein by reference).

The fact that heterologies in single stranded nucleic acid probes are tolerated allows for two things: first, the use of a heterologous consensus homology clamp that may target consensus functional domains of multiple genes, rather than a single gene, resulting in a variety of genotypes and phenotypes, and secondly, the introduction of alterations to the target sequence. Thus, in some embodiments, the recombinase coated single stranded nucleic acid probes have at least a portion or region having a sequence that is not present in the preselected endogenous targeted sequence(s) (i.e., a nonhomologous portion or mismatch) which may be as small as a single mismatched nucleotide, several mismatches, or may span up to about several kilobases or more of nonhomologous sequence.

When the single stranded nucleic acid probes are used to generate insertions or deletions in an endogenous nucleic acid target sequence, as is described herein, the use of two complementary single-stranded single stranded nucleic acid probes allows the use of internal homology clamps as depicted in the figures of PCT US Ser. No. 98/05223. The use of internal homology clamps allows the formation of stable deproteinized probe:target hybrids with homologous DNA sequences containing either relatively small or large insertions and deletions within a homologous DNA target. Without being bound by theory, it appears that these probe:target hybrids, with heterologous inserts in the single stranded nucleic acid probe, are stabilized by the re-annealing of single stranded nucleic acid probes to each other within the double-D-loop hybrid, forming a novel DNA structure with an internal homology clamp. Similarly stable double-D-loop hybrids formed at internal sites with heterologous inserts in the nucleic acid target sequences (with respect to the single stranded nucleic acid probe probe) are equally stable. Because single stranded nucleic acid probes are kinetically trapped within the duplex target, the multi-stranded DNA intermediates of homologous DNA pairing are stabilized and strand exchange is facilitated.

In a preferred embodiment, the length of the internal homology clamp (i.e. the length of the insertion or deletion) is from about 1 to 50% of the total length of the single stranded nucleic acid probe , with from about 1 to about 20% being preferred and from about 1 to about 10% being especially preferred, although in some cases the length of the deletion or insertion may be significantly larger. As for the consensus homology clamps, the complementarity within the internal homology clamp need not be perfect.

Thus, in a preferred embodiment, homologous recombination of the single stranded nucleic acid probes and nucleic acid target sequence will result in amino acid substitutions, insertions or deletions in the nucleic acid target sequences, potentially both within the consensus functional domain region and outside of it, for example as a result of the incorporation of PCR tags. This will generally result in modulated or altered gene function of the nucleic acid target sequence gene product, including both a decrease or elimination of function as well as an enhancement of function. Nonhomologous portions are used to make insertions, deletions, and/or replacements in a predetermined nucleic acid target sequences, and/or to make single or multiple nucleotide substitutions in a predetermined nucleic acid target sequence so that the resultant recombined sequence (i.e., a targeted recombinant endogenous sequence) incorporates some or all of the sequence information of the nonhomologous portion of the single stranded nucleic acid probe(s). Thus, the nonhomologous regions are used to make variant sequences, i.e. nucleic acid target sequence modifications. In this way, site directed modifications may be done in a variety of systems for a variety of purposes.

The nucleic acid target sequence may be disrupted in a variety of ways. The term “disrupt” as used herein comprises a change in the coding or non-coding sequence of an endogenous nucleic acid. In one preferred embodiment, a disrupted gene will no longer produce a functional gene product. In another preferred embodiment, a disrupted gene produces a variant gene product. Generally, disruption may occur by either the substitution, insertion, deletion or frame shifting of nucleotides.

In one embodiment, amino acid substitutions are made. This can be the result of either the incorporation of a non-naturally occurring consensus sequence into a consensus target, or of more specific changes to a particular sequence outside of the consensus sequence.

In one embodiment, the endogenous sequence is disrupted by an insertion sequence. The term “insertion sequence” as used herein means one or more nucleotides which are inserted into an endogenous gene to disrupt it. In general, insertion sequences can be as short as 1 nucleotide or as long as a gene, as outlined herein. For non-gene insertion sequences, the sequences are at least 1 nucleotide, with from about 1 to about 50 nucleotides being preferred, and from about 10 to 25 nucleotides being particularly preferred. An insertion sequence may comprise a polylinker sequence, with from about 1 to about 50 nucleotides being preferred, and from about 10 to 25 nucleotides being particularly preferred. Insertion sequence may be a PCR tag used for identification of the first gene.

In a preferred embodiment, an insertion sequence comprises a gene which not only disrupts the endogenous gene, thus preventing its expression, but also can result in the expression of a new gene product. Thus, in a preferred embodiment, the disruption of an endogenous gene by an insertion sequence gene is done in such a manner to allow the transcription and translation of the insertion gene. An insertion sequence that encodes a gene may range from about 50 bp to 5000 bp of cDNA or about 5000 bp to 50000 bp of genomic DNA. As will be appreciated by those in the art, this can be done in a variety of ways. In a preferred embodiment, the insertion gene is targeted to the endogenous gene in such a manner as to utilize endogenous regulatory sequences, including promoters, enhancers or a regulatory sequence. In an alternate embodiment, the insertion sequence gene includes its own regulatory sequences, such as a promoter, enhancer or other regulatory sequence etc.

Particularly preferred insertion sequence genes include, but are not limited to, genes which encode selection or reporter proteins. In addition, the insertion sequence genes may be modified or variant genes.

The term “deletion” as used herein comprises removal of a portion of the nucleic acid sequence of an endogenous gene. Deletions range from about 1 to about 100 nucleotides, with from about 1 to 50 nucleotides being preferred and from about 1 to about 25 nucleotides being particularly preferred, although in some cases deletions may be much larger, and may effectively comprise the removal of the entire consensus functional domain, the entire endogenous gene and/or its regulatory sequences. Deletions may occur in combination with substitutions or modifications to arrive at a final modified endogenous gene.

In a preferred embodiment, endogenous genes may be disrupted simultaneously by an insertion and a deletion. For example, some or all of an endogenous gene, with or without its regulatory sequences, may be removed and replaced with an insertion sequence gene. Thus, for example, all but the regulatory sequences of an endogenous gene may be removed, and replaced with an insertion sequence gene, which is now under the control of the endogenous gene's regulatory elements.

For many types of in vivo gene therapy to be effective, a significant number of cells must be correctly targeted, with a minimum number of cells having an incorrectly targeted recombination event. To accomplish this objective, the combination of: (1) a single stranded nucleic acid probe(s), (2) a recombinase (to provide enhanced efficiency and specificity of correct homologous sequence targeting), (3) an analog probe, and optionally (4) a cell-uptake component (to provide enhanced cellular uptake of the single stranded nucleic acid probe and/or analog probe), provides a means for the efficient and specific targeting of cells in vivo, making in vivo homologous sequence targeting, and gene therapy, practicable.

Several disease states may be amenable to treatment or prophylaxis by targeted alteration of heptocytes in vivo by homologous gene targeting. For example and not for limitation, the following diseases, among others not listed, are expected to be amenable to targeted gene therapy: hepatocellular carcinoma, HBV infection, familial hypercholesterolemia (LDL receptor defect), alcohol sensitivity (alcohol dehydrogenase and/or aldehyde dehydrogenase insufficiency), hepatoblastoma, Wilson's disease, congenital hepatic porphyrias, inherited disorders of hepatic metabolism, ornithine transcarbamylase (OTC) alleles, HPRT alleles associated with Lesch Nyhan syndrome, etc. Where targeting of hepatic cells in vivo is desired, a cell-uptake component consisting essentially of an asialoglycoprotein-poly-L- lysine conjugate is preferred. The targeting complexes of the invention which may be used to target hepatocytes in vivo take advantage of the significantly increased targeting efficiency produced by association of a single stranded nucleic acid probe with a recombinase which, when combined with a cell-targeting method such as that of WO Ser. No. 92/05250 and/or Wilson et al. (1992) J. Biol. Chem. 267:963, provide a highly efficient method for performing in vivo homologous sequence targeting in cells, such as hepatocytes.

In a preferred embodiment, the methods and compositions of the invention are used for gene inactivation. That is, in addition to correcting disease alleles, single stranded nucleic acid probes can be used to inactivate, decrease or alter the biological activity of one or more genes in a cell (or transgenic nonhuman animal). This finds particular use in the generation of animal models of disease states, or in the elucidation of gene function and activity, similar to “knock out” experiments.

These techniques may be used to eliminate a biological function; for example, a galT gene (alpha galactosyl transferase genes) associated with the xenoreactivity of animal tissues in humans may be disrupted to form transgenic animals (e.g. pigs) to serve as organ transplantation sources without associated hyperacute rejection responses. Alternatively, the biological activity of the wild-type gene may be either decreased, or the wild-type activity altered to mimic disease states. This includes genetic manipulation of non-coding gene sequences that affect the transcription of genes, including, promoters, repressors, enhancers and transcriptional activating sequences.

Once the specific target genes (including regulatory sequence) to be modified are selected, their sequences may be scanned for possible disruption sites (convenient restriction sites, for example). Plasmids are engineered to contain an appropriately sized gene sequence with a deletion, substitution or insertion in the gene of interest and at least one flanking homology clamp which substantially corresponds or is substantially complementary to an endogenous target DNA sequence. Vectors containing a single stranded nucleic acid probe sequence are typically grown in E. coli and then isolated using standard molecular biology methods, or may be synthesized as oligonucleotides. Direct targeted inactivation which does not require vectors may also be done. When using microinjection procedures it may be preferable to use a transfection technique with linearized sequences containing only modified target gene sequence and without vector or selectable sequences. The modified gene site is such that a homologous recombinant between the exogenous single stranded nucleic acid probe and the endogenous DNA target sequence can be identified by using carefully chosen primers and PCR, followed by analysis to detect if PCR products specific to the desired targeted event are present (Erlich et al., (1991) Science 252:1643, which is incorporated herein by reference). Several studies have already used PCR to successfully identify and then clone the desired transfected cell lines (Zimmer and Gruss, (1989) Nature 338:150; Mouellic et al., (1990) Proc. Nati. Acad. Sci. USA 87:4712; Shesely et al., (1991) Proc. Natl. Acad. Sci. USA 88:4294, which are incorporated herein by reference). This approach is very effective when the number of cells receiving exogenous single stranded nucleic acid probe(s) is high (i.e., with microinjection, or with liposomes) and the treated cell populations are allowed to expand to cell groups of approximately 1×10⁴ cells (Capecchi, (1989) Science 244:1288). When the target gene is not on a sex chromosome, or the cells are derived from a female, both alleles of a gene can be targeted by sequential inactivation (Mortensen et al., (1991) Proc. Nati. Acad. Sci. USA 88:7036).

In addition, the methods of the present invention are useful to add exogeneous DNA sequences, such as exogeneous genes or extra copies of endogeneous genes, to an organism. As for the above techniques, this may be done for a number of reasons, including: to alleviate disease states, for example by adding one or more copies of a wild-type gene or add one or more copies of a therapeutic gene; to create disease models, by adding disease genes such as oncogenes or mutated genes or even just extra copies of a wild-type gene; to add therapeutic genes and proteins, for example by adding tumor suppressor genes such as p53, Rb1, Wt1, NF1, NF2, and APC, or other therapeutic genes; to make superior transgenic animals, for example superior livestock; or to produce gene products such as proteins, for example for protein production, in any number of host cells. Suitable gene products include, but are not limited to, Rad51, alpha-antitrypsin, casein, hormones, antithrombin III, alpha glucosidase, collagen, proteases, viral vaccines, tissue plaminogen activator, monoclonal antibodies, Factors VIII, IX, and X, glutamic acid decarboxylase, hemoglobin, prostaglandin receptor, lactoferrin, calf intestine alkaline phosphatase, CFTR, human protein C, porcine liver esterase, urokinase, and human serum albumin.

For making transgenic non-human animals (which include homologously targeted non-human animals) embryonal stem cells (ES cells) and fertilized zygotes are preferred. In a preferred embodiment, embryonal stem cells are used. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embrvonic Stem Cells: A Practical Approach. E. J. Robertson, ed. (oxford: IRL Press), p. 71-112) may be used for homologous gene targeting. Other suitable ES lines include, but are not limited to, the E14 line (Hooper et al. (1987) Nature 326:292-295), the D3 line (Doetschman et al. (1985) J. Embryol. Exp. Morph. 87:21-45), and the CCE line (Robertson et al. (1986) Nature 323:445-448). The success of generating a mouse line from ES cells bearing a specific targeted mutation depends on the pluripotence of the ES cells (i.e., their ability, once injected into a host blastocyst, to participate in embryogenesis and contribute to the germ cells of the resulting animal).

Thus, in a preferred embodiment, the targeted sequence modification creates a sequence that has a biological activity or encodes a polypeptide having a biological activity. In a preferred embodiment, the polypeptide is an enzyme with enzymatic activity.

In addition to fixing or creating mutations involved in disease states, a preferred embodiment utilizes the methods of the present invention to create novel genes and gene products. Thus, fully or partially random alterations can be incorporated into genes to form novel genes and gene products, to produce rapidly and efficiently a number of new products which may then be screened, as will be appreciated by those in the art.

In a preferred embodiment, the compositions and methods of the invention are useful in site-directed mutagenesis techniques to create any number of specific or random changes at any number of sites or regions within a target sequence (either nucleic acid or protein sequence), similar to traditional site-directed mutagenesis techniques such as cassette mutagenesis and PCR mutagenesis. Thus, for example, the techniques and compositions of the invention may be used to generate site specific variants in any number of systems, including E. coli, Bacillus, Archebacteria, Thermus, yeast (Sacchromyces and Pichia), insect cells (Spodoptera, Trichoplusia, Drosophila), Xenopus, rodent cell lines including CHO, NIH 3T3 and primate cell lines including COS, or human cells, including HT1080 and BT474, which are traditionally used to make variants. The techniques can be used to make specific changes, or random changes, at a particular site or sites, within a particular region or regions of the sequence, or over the entire sequence.

In this and other embodiments, suitable target sequences include nucleic acid sequences encoding therapeutically or commercially relevant proteins, including, but not limited to, enzymes (proteases, recombinases, lipases, kinases, carbohydrases, isomerases, peptides tautomerases, nucleases etc.), hormones, receptors, transcription factors, growth factors, antibodies, cytokines, globin genes, immunosupppressive genes, tumor suppressors, oncogenes, complement-activating genes, milk proteins (casein, ″-lactalbumin, β-lactoglobulin, whey proteins, serum albumin), immunoglobulins, urine proteins, milk proteins, esterases, pharmaceutical proteins and vaccines.

The present invention is further directed to gene cloning comprising the rapid isolation of cDNA clones, which is facilitated by taking advantage of the catalytic function of the RecA enzyme and the hybridizing functions of the analog and single stranded nucleic acid probes as described above. By “cloning” herein is meant the isolation and amplification of a target sequence, which can be amplified, put into expression vectors, etc. as will be appreciated by those in the art.

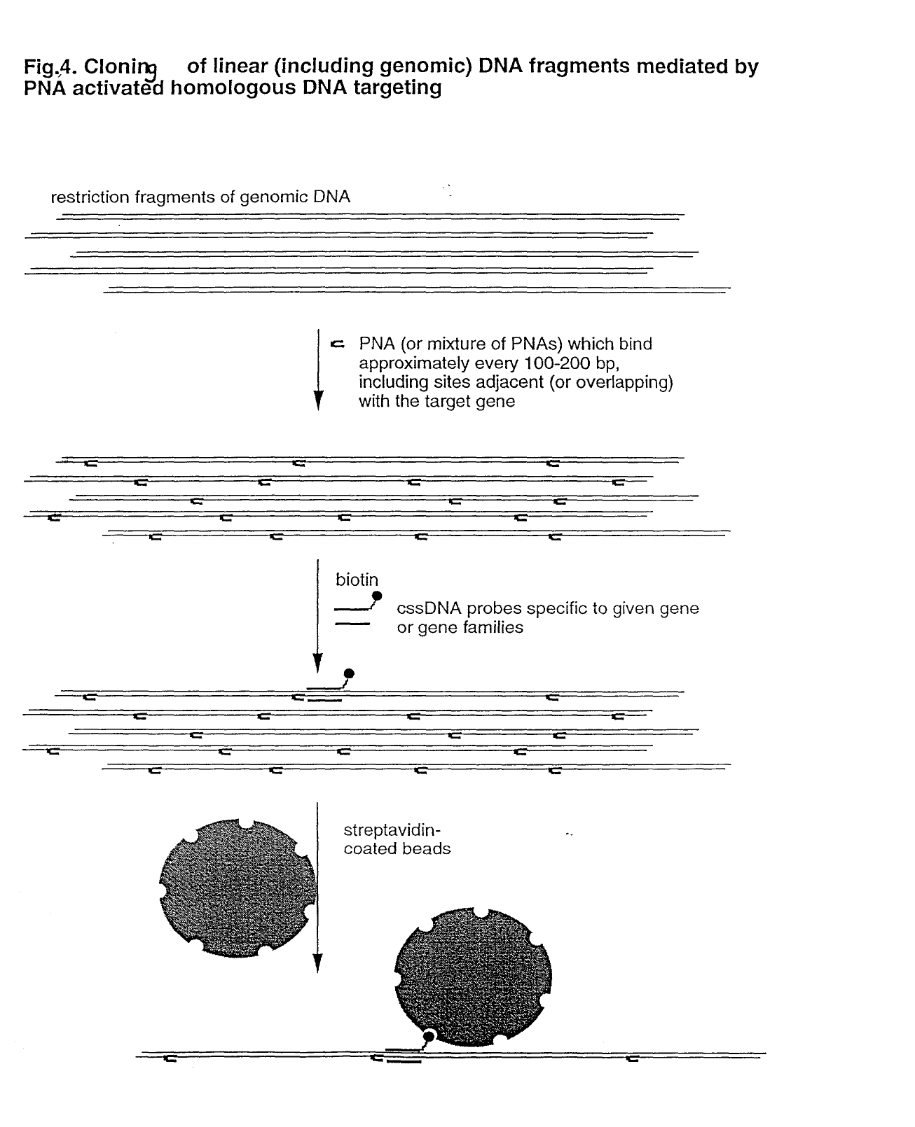

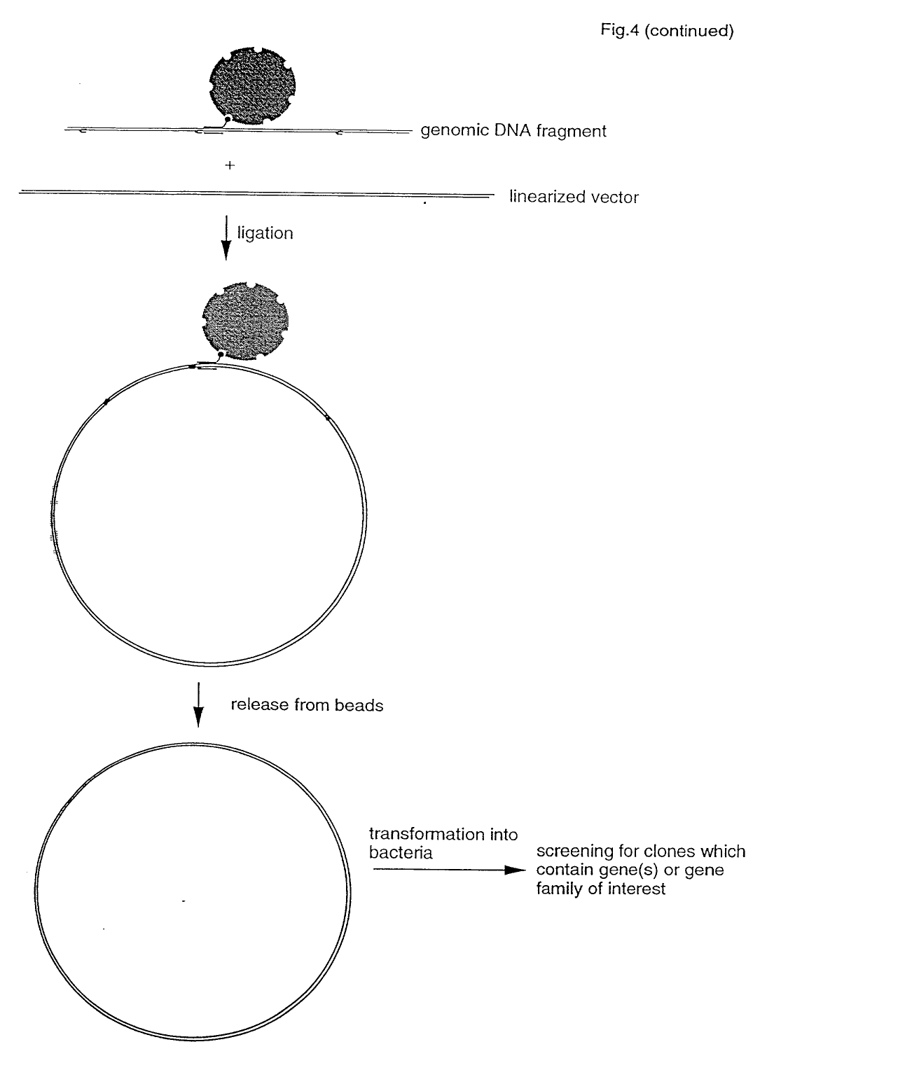

In a preferred embodiment, the analog recognition site is only 7-8 bases long. Such short analog probe-DNA complexes (at least 8-mers) are stable in the case of homopyrimidine bis-PNAs (Demidov et al. 1995, Proc. Natl. Acad. of Sci., 92,2637-2641; Faruqi et al., 1998, Proc. Natl. Acad. Sci., 95,1398-140). The recognition site for homopyrimidine PNAs of about 7 bases long would randomly occur every 100-200 base pairs. Thus, if the targeted sequence is more than 200 base pairs, in most cases it would have a very high probability of containing the recognition site for this PNA (FIG. 4). When most of the targeted sequences were unknown, a mixture of randomized homopyrimidine PNAs are used, which bind the targeted DNA approximately every 100-200 base pairs. Alternatively, pseudocomplimentary PNAs could be used for target activation.

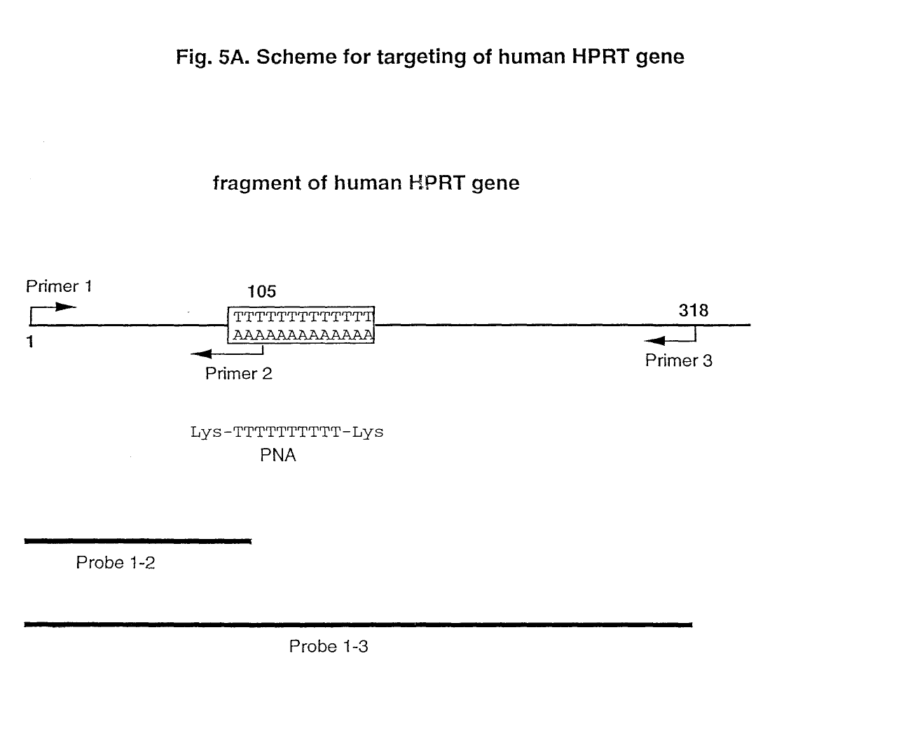

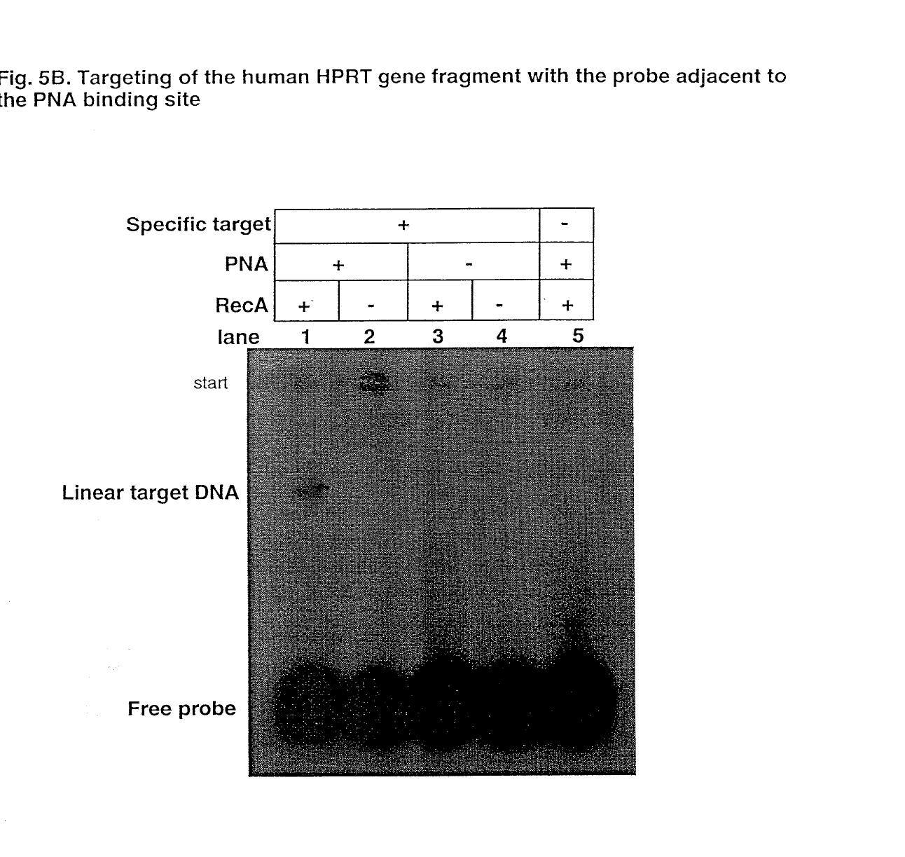

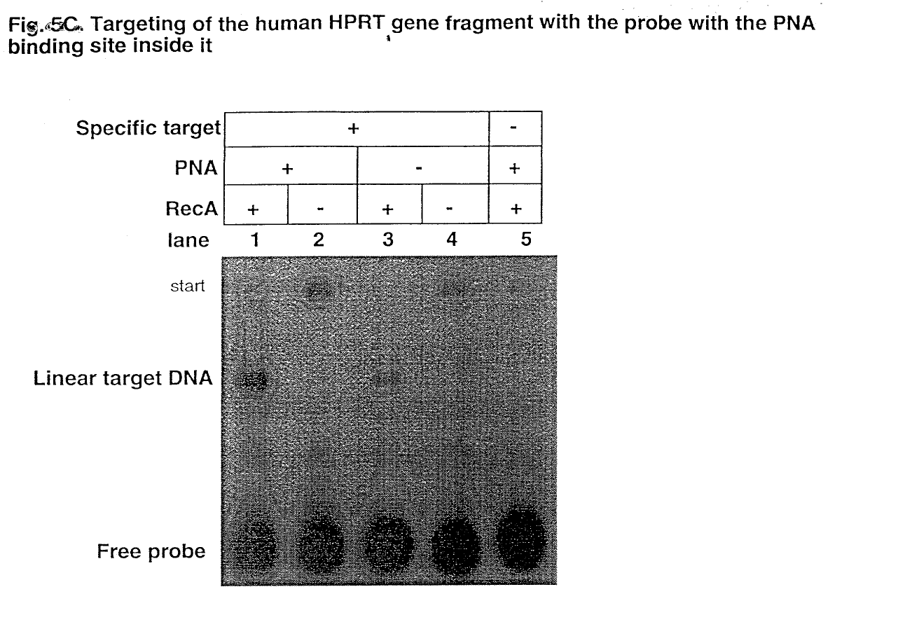

In a preferred embodiment, the recombinase coated single stranded nucleic acid probe, rather than PNA analog probe, is responsible for the specificity of the target recognition. The high specificity of PNA binding is not required for this strategy. This strategy of DNA targeting by PNA-mediated DNA opening is related to the P-D loop approach (Bukanov et al., Proc. Nati. Acad. Sci., 95,5516-5520), in which two PNA-binding sites are located close to each other. The binding of the PNAs opens the DNA region between them, providing a hybridization site for the recombinase coated single stranded nucleic acid analog probe. When RecA coated complementary single stranded nucleic acid analog probes are used, the presence of the PNA binding sites on both ends of the target sequence is not required. Moreover, experimental data (Fig.5A, 5B and 5C) indicates that PNA accelerates targeting not only when it binds to an adjacent site to the target sequence, but also when it binds within the target sequence. This further increases the flexibility and utility of his approach in applications with homologous DNA targeting.

Thus, in a preferred embodiment, the present invention provides methods for isolating new members of gene families comprising introducing single stranded nucleic acid probes comprising consensus homology clamps and at least one purification tag, preferably biotin, to a mix of nucleic acid, such as a plasmid cDNA library or a cell, and then utilizing the purification tag to isolate the gene(s). The exact methods will depend on the purification tag; a preferred method utilizes the attachment of the binding ligand for the tag to a bead, which is then used to pull out the sequence. Alternatively anti-recA antibodies could be used to capture recA-coated probes. The genes are then cloned, sequenced, and reassembled if necessary, as is well known in the art.

Accordingly, the compositions of the invention may be added to cell lysates or cDNA libraries according to methods well known in the art.

The compositions and methods of the invention are further directed to the detection of nucleic acid target sequences. The nucleic acid target sequence comprises a position for which sequence information is desired.

The present invention utilizes analog probes and recombinase proteins to facilitate the hybridization of single stranded nucleic acid probes to the nucleic acid target sequence. As described above, the analog probe forms nucleation site that allows the recombinase coated single stranded nucleic acid probes of the invention to bind to the nucleic acid target sequence. The recombinase enzyme then catalyzes the pairing and strand exchange between the target nucleic acid sequence and the single stranded nucleic acid probe. This pairing leads to the formation of multistranded nucleic acid hybrids which can subsequently be detected by known methods.

As is known in the art, there are a number of techniques that can be used to detect or determine the identity of a base at a particular location in a target nucleic acid, including, but not limited to, the use of temperature, competitive hybridization of perfect and imperfect single stranded nucleic acid probes to the target sequence, sequencing by synthesis, for example using single base extension techniques (sometimes referred to as “minisequencing”), the oligonucleotide ligase amplification (OLA) reaction, rolling circle amplification (RCA), allelic PCR, competitive hybridization and Invader™ technologies.

In a preferred embodiment, the methods of the invention are used to detect pathogens such as bacteria.

In a preferred embodiment, the target sequences include rRNA.

If required, the nucleic acid target sequence is prepared using known techniques. For example, the sample may be treated to lyse the cells, using known lysis buffers, electroporation, etc., with purification and/or amplification as needed, as will be appreciated by those in the art. In addition, techniques to increase the amount or rate of hybridization can also be used.

The methods of the invention find particular use in genotyping assays, i.e. the detection of particular nucleotides at specific positions, although as will be appreciated by those in the art, amplification and/or quantification need not necessarily occur to do genotyping.

Accordingly, the compositions and methods of the present invention are used to identify the nucleotide(s) at a detection position within the nucleic acid target sequence.

In a preferred embodiment, kits containing the compositions of the invention are provided. The kits include the compositions, particularly those of libraries or pools of degenerate single stranded nucleic acid probes, analog with any number of reagents or buffers, including recombinases, buffers, salts, ATP, etc.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

PNA activated targeting of linear plasmid DNA containing the human HPRT gene by RecA-coated complementary single-stranded (css) probes (See FIG. 5A, 5B and 5C): Plasmid pHp3A-pUC containing a 530 bp fragment of human HPRT gene was cloned into the EcoR1-Hindil site of the vector pUC 18 (See FIG. 5A). Complementary strand probes (1-2) and (1-3) were obtained by PCR from the pHp3A-pUC plasmid using the primers shown in FIG. 5A. The human HPRT fragment contains sequence (dT)/(dA) 13, which provides an ideal binding site for the PNA lys-T,₁₀- lys. No other (dT)/(dA)-stretches longer than 5 bp are present within the HPRT fragment. The probe (1-2) has a three bp overlap with the PNA binding site. T10 was used instead of T13PNA because the solubility of the PNA decreased with increased length. Thus, PNA could occupy four different positions within the site, with the overlap with the (1-2) probe varying from 0 to 3. The probe (1-3) contained the PNA binding site within it.

As a specific target DNA, the pHp3ApUC plasmid linearized by the restriction enzyme, Sca I, was used. As a heterologous control, pUCI9 linearized by the restriction enzyme Sca I was used.

Briefly, the target plasmid was incubated with 10 μM of PNA in 9 mM Tris HC1 (pH 8), 0.09 mM EDTA for about 3 hours at 37° C. Next, it was mixed with radioactively labeled RecA protein coated css probes, incubated for 2 hours at 37° C., deproteinized by the addition of SDS, and loaded on an agarose gel. The probe-target hybrid formed was detected by the radioactive signal at the position of the target DNA on the gel. The unbound free probe migrates much faster on the gel than the probe-target hybrids. The conditions for RecA protein coating of the complementary single strand probes in the targeting reaction were similar to the conditions used by Sena and Zarling (1993, in Nature Genet., 3,365-372).

FIG. 5B and 5C show the results of these experiments using probes (1-2) and (1-3) respectively. We observed, that in both cases, PNA strongly increased the yield of the hybrids (See lane 1 of FIG. 5 B and C).

We claim:

1. A composition comprising:
a) an analog probe; and
b) a first recombinase coated single stranded nucleic acid probe.

2. A composition according to claim 1 further comprising a second recombinase coated single stranded nucleic acid probe which is substantially complementary to said first probe.

3. A composition according to claim 1 wherein said analog probe comprises peptide nucleic acid.

4. A composition according to claim 1 wherein said analog probe is a fusion sequence comprising nucleoside analogs and naturally occurring nucleosides.

5. A composition according to claim 4 wherein said nucleoside analog comprises at least one peptide nucleoside.

6. A composition according to claim 1 wherein said first single stranded nucleic acid probe is DNA.

7. A composition according to claim 1 wherein said recombinase is a species of a prokaryotic recombinase.

8. A composition according to claim 7 wherein said prokaryotic recombinase is a recA recombinase.

9. A composition according to claim 1 wherein said recombinase is a species of eukaryotic recombinase.

10. A composition according to claim 9 wherein said recombinase is a Rad51 recombinase.

11. A composition according to claim 9 wherein said recombinase is a complex of recombinase proteins.

12. A method comprising:
a) providing a sample comprising double stranded nucleic acid target sequence;
b) activating nucleic acid targeting using an analog probe; and
c) hybridizing to said double stranded nucleic acid target sequence at least a first recombinase coated single stranded nucleic acid probe comprising a homology clamp that is substantially complementary to one strand of said target nucleic acid sequence.

13. A method according to claim 12 further comprising a second recombinase coated single stranded nucleic acid probe, which hybridizes to said double stranded nucleic acid target sequence and is substantially complementary to said first recombinase coated single stranded nucleic acid probe.

14. A method according to claim 12 wherein at least one of said first and second probes comprises at least one alteration as compared to said target sequence, and wherein said method further comprises altering said target sequence by homologous recombination with at least one of said probes.

15. A method according to claim 14 wherein both first and second recombinase coated single stranded nucleic acid probes have said alteration.

16. A method according to claim 14 wherein said alteration comprises a substitution mutation of at least on nucleic acid.

17. A method according to claim 14 wherein said alteration comprises a deletion mutation of at least on nucleic acid.

18. A method according to claim 14 wherein said alteration comprises an insertion of said at least one nucleic acid.

19. A method according to claim 12 wherein said activating analog probe creates a nucleation site for hybridization of said first recombinase coated single stranded nucleic acid to said nucleic acid target sequence.

20. A method according to claim 19 wherein said nucleation site is a D-loop structure.

21. A method according to claim 20 wherein said D-loop structure is either a single or a double D-loop structure.

22. A method according to claim 19 wherein said activating analog probe forms a hybridization complex with some portion of said nucleic acid target sequence.

23. A method according to claim 22 wherein said hybridization complex is at least as stable as a non-analog nucleic acid hybridization complex.

24. A method according to claim 12 wherein said activating analog probe facilitates the production of double stranded gaps in the target nucleic acid sequence which are repaired by said recombinase coated single stranded nucleic acid probes.

25. A method according to claim 24 wherein said activating analog probe forms a hybridization complex with some portion of said nucleic acid target sequence.

26. A method according to claim 25 wherein said hybridization complex is at least as stable as a non-analog nucleic acid hybridization complex.

27. A method according to claim 12 wherein said analog probe comprises peptide nucleic acid.

28. A method according to claim 12 wherein said recombinase is a species of a prokaryotic recombinase.

29. A method according to claim 28 wherein said prokaryotic recombinase is a RecA recombinase.

30. A method according to claim 12 wherein said recombinase is a species of eukaryotic recombinase.

31. A method according to claim 30 wherein said recombinase is a Rad51 recombinase.

32. A method according to claim 30 wherein said recombinase is a complex of recombinase proteins.

33. A method according to claim 13 wherein at least one of said first and second probes comprises a purification tag, and said method further comprises separating said probes and said target sequence from said sample using said tag.

34. A method according to claim 33 further comprising inserting the nucleic acid target sequence into a cloning vector.

35. A method according to claim 33 wherein said purification tag comprises biotin.
